(19) United States
(12) Patent Application Publication
(10) Pub. No.: US 2025/0375129 A1
(43) Pub. Date: Dec. 11, 2025

(54) USE OF BLINK REFLEX FOR ATTENTION-DEFICIT/HYPERACTIVITY DISORDER (ADHD) RESPONSE MONITORING AND/OR TREATMENT SELECTION

(71) Applicant: Blinktbi, Inc., Charleston, SC (US)

(72) Inventors: Jennifer FIORINI, Charleston, SC (US); Dena GARNER, Charleston, SC (US); Douglas CARNES, Charleston, SC (US); Andrew MASSEY, Charleston, SC (US); Jeffrey RILEY, Charleston, SC (US)

(21) Appl. No.: 18/837,449

(22) PCT Filed: Feb. 10, 2023
(86) PCT No.: PCT/US2023/012797

§ 371 Date: Aug. 9, 2024

Related U.S. Application Data
(60) Provisional application No. 63/309,222, filed on Feb. 11, 2022.

(51) Int. Cl. A61B 5/16; A61B 5/00; A61B 5/11

(52) U.S. Cl. CPC A61B 5/168; A61B 5/0053; A61B 5/1103; A61B 5/4833; A61B 5/4842; A61B 5/6803

(57) ABSTRACT

The disclosure relates to, in part, a method of diagnosing whether a patient is afflicted with attention-deficit/hyperactivity disorder (ADHD) and/or selecting ADHD treatment for a patient afflicted with ADHD and/or monitoring response of ADHD symptoms in patient to an ADHD treatment.

### FIELD

The present disclosure provides methods and devices for attention-deficit/hyperactivity disorder (ADHD) response, diagnosis, monitoring, and/or treatment selection in a patient.

### RELATED APPLICATIONS

The application is a National Stage of PCT Application No. PCT/US2023/012797, filed Feb. 10, 2023, which claims the benefit of, and priority to, U.S. Provisional Application No. 63/309,222, filed Feb. 11, 2022, the contents of which are hereby incorporated by reference in their entirety.

### BACKGROUND

Attention-deficit/hyperactivity disorder (ADHD) is a developmental disorder distinguished by symptoms of inattention, hyperactivity, and impulsivity. Although ADHD is one of the most frequently diagnosed psychological disorders in childhood long-term studies have demonstrated that symptoms can be maintained into adulthood. Adult ADHD has now been recognized as a valid clinical entity that is associated with profound psychosocial and cognitive impairments. Currently, the tools and methods for diagnosing whether a patient is afflicted with ADHD are unreliable. There is a need for effective methods of diagnosing, predicting, and/or treating ADHD in a patient. Moreover, there is a need for effective methods of selecting treatment and/or monitoring response to treatment in patient afflicted with ADHD.

### SUMMARY

Accordingly, the present disclosure provides a method of diagnosing whether a patient is afflicted with attention-deficit/hyperactivity disorder (ADHD), comprising: (a) measuring a blink reflex in a patient suspected to be afflicted with ADHD, the measurement comprising: (i) applying a non-electrical stimulus to an eye of the patient to induce a blink reflex and (ii) assessing one or more blink-associated parameters; and (b) diagnosing the patient as afflicted with ADHD or not afflicted with ADHD by comparing the one or more blink-associated parameters to equivalent one or more blink-associated parameters from a patient who is not afflicted with ADHD.

In aspects, the present disclosure provides a method of diagnosing and treating a patient afflicted with ADHD, comprising: (a) measuring a blink reflex in a patient suspected to be afflicted with ADHD, the measurement comprising: (i) applying a non-electrical stimulus to an eye of the patient to induce a blink reflex and (ii) assessing one or more blink-associated parameters; (b) diagnosing the patient as afflicted with ADHD or not afflicted with ADHD by comparing the one or more blink-associated parameters to equivalent one or more blink-associated parameters from a patient who is not afflicted with ADHD; and (c) administering an effective amount of an ADHD treatment, optionally a medication, to a patient diagnosed as afflicted with ADHD.

In aspects, the present disclosure provides a method of selecting an ADHD treatment for a patient afflicted with ADHD, comprising: (a) measuring a blink reflex in the patient afflicted with ADHD, the measurement comprising: (i) applying a non-electrical stimulus to an eye of the patient to induce a blink reflex and (ii) assessing one or more blink-associated parameters; and (b) selecting an ADHD treatment for the patient afflicted with ADHD by comparing the one or more blink-associated parameters to equivalent one or more blink-associated parameters from the patient when receiving the ADHD treatment.

In aspects, the present disclosure provides a method of assessing response of an ADHD patient to an ADHD treatment, comprising (a) measuring a blink reflex in the patient afflicted with ADHD and who has undergone or is undergoing treatment with the ADHD treatment, the measurement comprising: (i) applying a non-electrical stimulus to an eye of the patient to induce a blink reflex and (ii) assessing one or more blink-associated parameters; and (b) determining a response to the ADHD treatment by comparing the one or more blink-associated parameters to equivalent one or more blink-associated parameters from the patient before receiving the ADHD treatment.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 depicts a non-limiting exemplary diagrammatic representation of a pilot study in humans for assessing the clinical significance of measuring blink reflex response in a cohort of patients diagnosed with ADHD in comparison to a non-ADHD control group.

FIG. 2 depicts a non-limiting exemplary tabular representation of blink reflex response parameters in a cohort of patients diagnosed with ADHD (n=24) in comparison to a non-ADHD control group (n=17, before +after measurements represented as n=34). Statistical significance was determined at p<0.05 using a student's t-test.

FIG. 3 depicts a non-limiting exemplary tabular representation of the mean changes of blink reflex response parameters in a cohort of patients diagnosed with ADHD (n=24) in comparison to a non-ADHD control group (n=17) prior to taking ADHD medication and 1 hour post-medication. Statistical significance was determined at p<0.05 using student's t-test.

FIG. 4 depicts a non-limiting exemplary diagrammatic representation of the statistical analyses between ADHD (n=24) and control (n=18) groups for blink reflex response parameters for mean delta 30 (measured as time in milliseconds (ms) between ipsilateral eye and contralateral eye movement), mean initial eyelid velocity (measured in pixels/ms), and mean maximal eyelid closing velocity (measured in pixels/ms). Data shown as mean±S.D. Statistical significance is set at p<0.05 using student's t-test.

FIG. 5 depicts a non-limiting exemplary diagrammatic representation of the statistical analysis of eyelid excursion (measured in pixelate distance traveled from tonic lid position to closed position) between ADHD (n=24) and control (n=18) groups. Data shown as mean±S.D. Statistical significance is set at p<0.05 using student's t-test.

FIG. 6 depicts a non-limiting exemplary diagrammatic representation of the difference in the mean time to open between n=24) and control (n=18) groups for time to open eyelid. Data shows mean difference±95% confidence interval (CI) for the ADHD group before and after medication as compared to the control group, illustrating the difference in the time required for opening the eyelid (measured as time in ms for eyelid to travel from closed position to tonic lid position).

FIG. 7 depicts a non-limiting exemplary diagrammatic representation of the differences in eyelid latency parameters between the ADHD group (n=24) before and after medication relative to the control group (n=18). The data shows no statistically significant difference between the ADHD group pre medication (Pre Med) and after medication (Post Med) and the control group for upper eyelid and lower eyelid latency (measured in ms). Data shown as mean±S.D. Statistical significance is set at p<0.05.

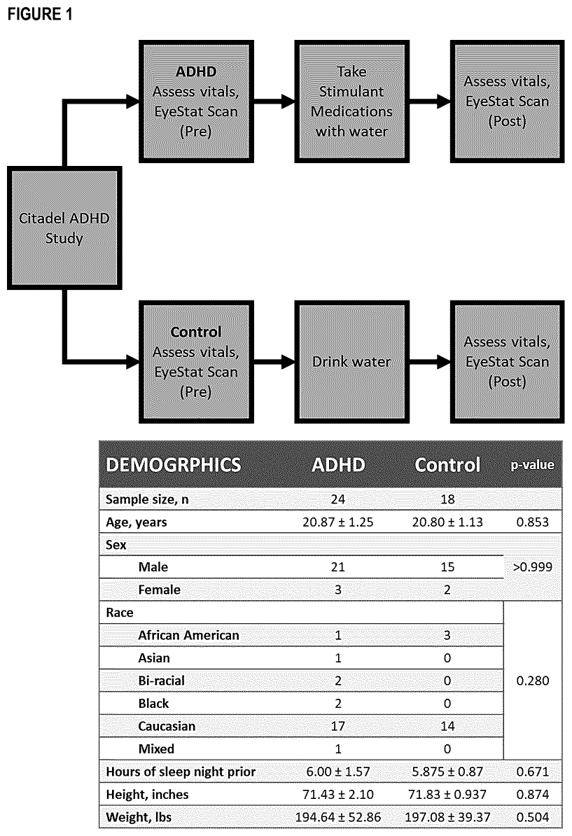

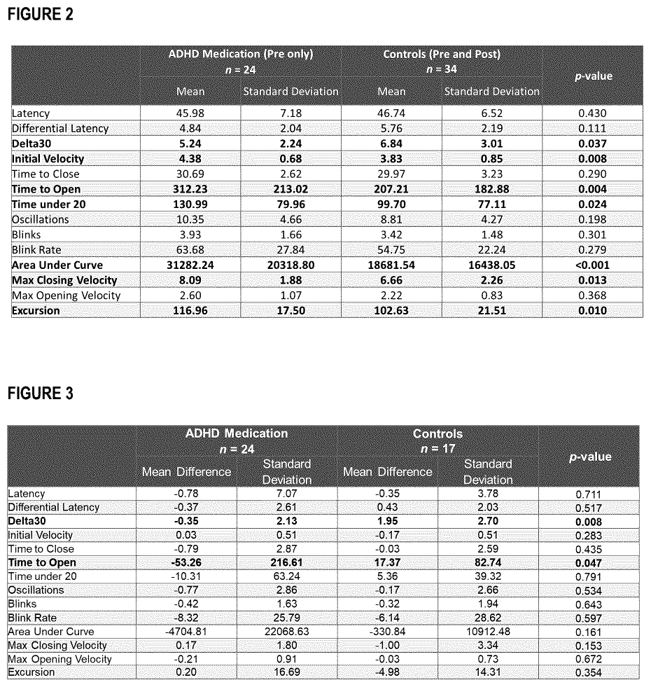

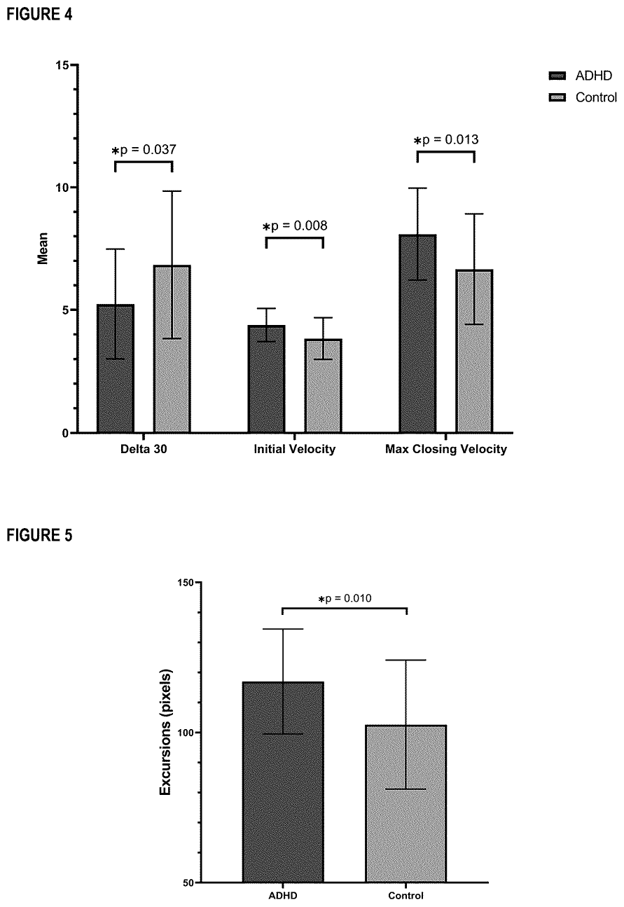

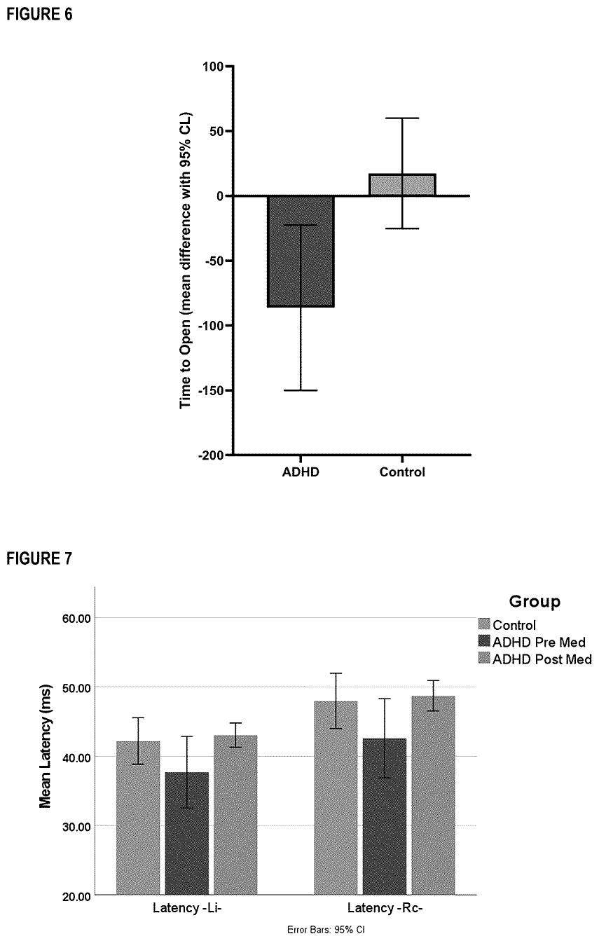

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

The blink reflex involves two cranial nerves, the trigeminal (V) and facial (VII) nerves, routing through the medulla and pons in the brainstem. The blink reflex is a true reflex, with a sensory afferent limb (V), intervening synapse, and a motor efferent limb (VII). Damage or disorder to these tracts (i.e., from neurodegeneration or trauma) will cause a change in the blink reflex.

A physiological biomarker of brain function used in assessments, diagnosis, and treatment of ADHD herein, in embodiments, includes measurement of one or more parameters of the blink reflex response. In embodiments, the blink response provides a quantitative, objective analysis of ADHD symptoms and provides an objective diagnostic for diagnosing ADHD, gauging treatment effectiveness and adherence to ADHD medication.

The present disclosure provides a method of diagnosing whether a patient is afflicted with attention-deficit/hyperactivity disorder (ADHD), comprising: (a) measuring a blink reflex in a patient suspected to be afflicted with ADHD, the measurement comprising: (i) applying a non-electrical stimulus to an eye of the patient to induce a blink reflex and (ii) assessing one or more blink-associated parameters; and (b) diagnosing the patient as afflicted with ADHD or not afflicted with ADHD by comparing the one or more blink-associated parameters to an equivalent one or more blink-associated parameters from a patient who is not afflicted with ADHD.

In aspects, the present disclosure provides a method of diagnosing and treating a patient afflicted with ADHD, comprising: (a) measuring a blink reflex in a patient suspected to be afflicted with ADHD, the measurement comprising: (i) applying a non-electrical stimulus to an eye of the patient to induce a blink reflex and (ii) assessing one or more blink-associated parameters; (b) diagnosing the patient as afflicted with ADHD or not afflicted with ADHD by comparing the one or more blink-associated parameters to equivalent one or more blink-associated parameters from a patient who is not afflicted with ADHD; and (c) administering an effective amount of an ADHD treatment, optionally a medication, to a patient diagnosed as afflicted with ADHD.

In aspects, the present disclosure provides a method of selecting an ADHD treatment for a patient afflicted with ADHD, comprising: (a) measuring a blink reflex in the patient afflicted with ADHD, the measurement comprising: (i) applying a non-electrical stimulus to an eye of the patient to induce a blink reflex and (ii) assessing one or more blink-associated parameters; and (b) selecting an ADHD treatment for the patient afflicted with ADHD by comparing the one or more blink-associated parameters to equivalent one or more blink-associated parameters from the patient when receiving the ADHD treatment.

In embodiments, the present disclosure provides a method of assessing response of an ADHD patient to an ADHD treatment, comprising (a) measuring a blink reflex in the patient afflicted with ADHD and who has undergone or is undergoing treatment with the ADHD treatment, the measurement comprising: (i) applying a non-electrical stimulus to an eye of the patient to induce a blink reflex and (ii) assessing one or more blink-associated parameters; and (b) determining a response to the ADHD treatment by comparing the one or more blink-associated parameters to equivalent one or more blink-associated parameters from the patient before receiving the ADHD treatment.

In embodiments, methods herein include an assessment of the response of an ADHD patient to an ADHD treatment by performing a dosing titration of the treatment to identify an effective dose. Dosing titration, in embodiments, includes providing one or more different doses (e.g., starting from a minimum dose, followed by a dose escalation), and measuring one or more blink reflex response parameters to determine if the dosage is appropriately improving, or otherwise normalizing, the parameters towards a healthy parameter measure (e.g., the blink reflex response of a person without ADHD).

In embodiments, the method further comprises prescribing an ADHD treatment as a function of the blink reflex parameters. In embodiments, prescribing comprises changing a prescription from a first ADHD medication to a different ADHD medication.

In embodiments, methods herein are used to determine patient adherence to one or more ADHD treatments, e.g., as described herein. In embodiments, the method further comprises determining a level of adherence to ADHD treatment as a function of the blink reflex. For example, in non-limiting embodiments, an ADHD-diagnosed patient who has been prescribed a treatment can be assessed at one or more times after prescription to determine if blink reflex parameters are improving, or otherwise normalizing, relative to a patient adhering to a cognate treatment. In embodiments, ADHD treatments include AMP (e.g., Amphetamine salts, Adderall, Vyvanse), ATX (e.g., Atomoxetine based, non-stimulant), MPH (e.g., methylphenidate), and/or behavioral therapy.

In embodiments, the methods of the present disclosure provide information that can be used for pre-emptive treatment to avoid the onset of one or more symptoms of ADHD (e.g., without limitation, inattention, impulsivity and hyperactivity). In embodiments, the methods of the present disclosure provide relevant information for preventing further exacerbation of one or more symptoms of ADHD (e.g., without limitation, inattention, impulsivity and hyperactivity). In embodiments, the methods of the present disclosure provide relevant information for preventing a patient from reaching higher scores of a rating scale (e.g., without limitation, typically on a point scale of 0-3 or 4 and usually, 0 means never, whereas 3 or 4 means very often) when scoring behaviors. In embodiments, the rating scale is selected from: Child Behavior Checklist (CBCL), Conners-Wells' Adolescent Self-Report Scale, Swanson, Nolan, and Pelham-IV Questionnaire (SNAP-IV), National Institute for Children's Health Quality (NICHQ) Vanderbilt Assessment Scale, and Conners Comprehensive Behavior Rating Scale (CBRS). In embodiments, the rating scale is selected from: Adult ADHD Self-Report Scale (ASRS v1.1), Adult ADHD Clinical Diagnostic Scale (ACDS) v1.2, Brown Attention-Deficit Disorder Symptom Assessment Scale (BADDS) for Adults, and ADHD Rating Scale-IV (ADHD-RS-IV). In embodiments, typical questions and scoring system may probe the extent of excessive talking or fidgeting to gauge hyperactivity. In embodiments, typical questions and scoring system may probe the extent of interrupting to gauge impulsivity. In embodiments, the methods of the present disclosure provide relevant information for preventing a patient from experiencing higher severity of inattention, hyperactivity, and/or impulsiveness.

In embodiments, the diagnosing comprises: diagnosis of disease onset; determination of disease stage and/or severity; determination of disease progress; and/or determination of cognitive state and/or mental capacity.

In embodiments, the method is carried out remotely, optionally with a telehealth device. In embodiments, the method is adapted for point-of-care application.

In embodiments, the information collected on the blink reflex response is used to predict correlation of ADHD to the occurrence of a future disease and/or disorder of the central nervous system.

In embodiments, an information collected further allows for assessing the therapeutic efficacy of pharmaceutical and/or medical and/or surgical treatment modalities.

In embodiments, the methods of the present disclosure provide information that may inform modifying the patient's treatment (e.g., without limitation, changing treatment (e.g. adding or removing one or more ADHD treatments), altering dosage of the ADHD treatment(s), and/or altering the ADHD treatment regimen (e.g. altering frequency and/or duration of dosing).

In embodiments, the method of the present disclosure measures latency until physiological response to stimulus. In embodiments, the blink reflex correlates with functionality of one or more of the trigeminal and facial nerves.

In embodiments, the stimulus is non-electrical. In embodiments, the non-electrical stimulus is one or more physical stimuli. In embodiments, the one or more physical stimuli are mechanical, visual, and/or acoustic. In embodiments, the one or more physical stimuli are light, heat, and/or sound. In embodiments, the one or more physical stimuli are a puff of air, a flash of light, a noise, and/or a vibration. In embodiments, wherein the stimulus is a sound, the stimulus triggers blink reflex when the sound is about 40 to about 60 dB greater than the minimum dB level for the patient. In embodiments, the puff of air is a short light gust of air. In embodiments, the puff of air is a tactile stimulus. In embodiments, the air is or comprises compressed air. In embodiments, the compressed air is delivered to the user's and/or patient's eye at a predetermined pressure. In embodiments, the puff of air has a pressure of about 3.75 psi (˜25 kPa) to about 60 psi˜415 kPa) (e.g. about 5 kPa, or about 10 kPa, or about 15 kPa, or about 20 kPa, or about 25 kPa). In embodiments, the puff of air is delivered at a duration of about 100 ms and a peak pressure of about 60 psi (˜415 kPa). In embodiments, the air is or comprises carbon dioxide. In embodiments, the air is or comprises nitrogen. In embodiments, the air is or comprises oxygen. In embodiments, the air puff stimulus is delivered from an air source. In embodiments, air source comprises an air compressor. In embodiments, air source comprises an air compression system. In embodiments, air source comprises a tank of industrial air. In embodiments, the air is purified air, usually food grade versions of air, CO₂, nitrogen, etc, are used.

In embodiments, the air compression system may include a gas source, holding tank, power source, and regulator. In embodiments, the holding tank may be coupled to gas source and may receive gas outputted from gas source. In embodiments, the regulator controls gas source and outputs gas from gas source into holding tank prior to being release to the user or patient to initiate a blink reflex. In embodiments, the regulator may be a valve and may be coupled to power source and gas source. In embodiments, the regulator may be coupled to gas source or may be integrally formed with gas source.

In embodiments, the regulator may be configured to reduce the pressure of the gas outputted by gas source. For example, the regulator may be configured to step down the pressure outputted from gas source to under 100 PSI. In embodiments, the pressure of the gas outputted by gas source is approximately 900 PSI and regulator is configured to step down the pressure from gas source to less than 100 PSI. However, the regulator may be configured to step down the pressure to less than about 500 PSI, less than about 400 PSI, less than about 200 PSI, or less than about 50 PSI. In embodiments, the regulator is configured to allow certain amounts of pressure into holding tank that can then be released at different pressures to initiate one or more blink reflexes. In embodiments, the regulator may be configured to allow for different outputs of pressure of gas for different users or patients of blink reflex device.

In embodiments, the regulator may be configured to fine tune the pressure of gas outputted by blink reflex that initiates the blink reflex of the user or patient. In embodiments, the regulator may be configured to output gas (via gas source) at the minimum pressure required to initiate a blink reflex. For example, regulator may be configured to output gas at a pressure of less than about 50 PSI. In embodiments, the regulator is configured to received compressed gas from gas source and output compressed gas from blink reflex device at a predetermined pressure for a predetermined duration. For example, the regulator may be configured to output compressed gas at a pressure of about 3 PSI to about 60 PSI or at a pressure of about 30 PSI to about 50 PSI. However, regulator may be configured to output gas at a pressure of about 10 PSI to about 100 PSI, about 20 PSI to about 80PSI, or about 40 PSI to about 60 PSI. In embodiments, regulator outputs a first puff of compressed gas at a first pressure and then a second puff of compressed air at a second pressure. In embodiments, the first pressure may be less than the second pressure. In embodiments, the regulator outputs compressed gas at the first pressure for one or more puffs and then outputs compressed gas at the second pressure for one or more puffs.

In embodiments, the regulator is to be configured to determine the minimum pressure required to initiate a blink reflex. During use, the regulator may initially output compressed gas at a low pressure and gradually increase the pressure of compressed gas outputted until a blink reflex is detected. In embodiments, the regulator increases the pressure of compressed gas by about 1 to about 3 PSI. However, regulator may increase the pressure of compressed gas by about 0.5 PSI to about 5 PSI, about 1 PSI to about 3 PSI, or less than about 0.5 PSI.

In embodiments, the stimulus is delivered to the desired location, e.g., without limitation, on the face of the subject. In embodiments, the stimulus is delivered to the forehead. In embodiments, the stimulus is delivered to the cheek. In embodiments, the compressed air is applied across the left eye, the right eye, and/or both eyes. In embodiments, the compressed air is applied across the eye. In embodiments, the non-electrical stimulus is applied to the canthus, outer canthus, inner canthus, lateral canthus, or medial canthus. In embodiments, applying the non-electrical stimulus to the canthus or the outer canthus produces a robust blink reflex. In embodiments, the non-electrical stimulus is not applied directly onto the cornea.

In embodiments, the blink reflex is measured with a high-speed camera. In embodiments, the blink reflex is measured by capturing and analyzing high-definition images. In embodiments, the blink reflex is measured by capturing and analyzing high-definition video. In embodiments, the present measurement, e.g. via a device for measuring, capturing, and analyzing high-definition images and/or video is further capable of distinguishing between the desired blink and undesired blink. In embodiments, the desired blink is triggered as a result of a blink reflex. In embodiments, the desired blink is triggered by a stimulus (e.g., without limitation, a puff of air, light, heat, and/or sound). In embodiments, the undesired blink is a false-positive blink, false-negative blink, or an abnormal blink.

In embodiments, the desired blink is or comprises an induced blink. In embodiments, the undesired blink is or comprises spontaneous and/or voluntary blink. In embodiments, the present measurement, e.g. via a device for measuring, capturing, and analyzing high-definition images and/or video omits or flags the undesired blink (e.g., without limitation, a false-positive blink, a false-negative blink, or an abnormal blink) from the final results generated. In embodiments, the present measurement, e.g., via a device for measuring, capturing, and analyzing high-definition images, includes the undesired blink (e.g., without limitation, a spontaneous and/or voluntary blink) in the final results generated. In embodiments, the present measurement, e.g., via a device for measuring, capturing, and analyzing high-definition images, includes the desired blink (e.g., without limitation, an induced blink) in the final results generated.

In embodiments, the blink reflex is not measured with an EMG sensor. In embodiments, the blink reflex is measured with a blink reflex monitoring device. In embodiments, the blink reflex monitoring device is a wearable device or a handheld device or can be placed on a tripod, on a desk, or the like. In embodiments, the blink reflex monitoring device is simply placed on a stool or a chair. In embodiments, the blink reflex monitoring device is simply hung on a wall such that the device is able to monitor and record the blink reflex of the user's and/or patient's eye. In embodiments, the blink reflex monitoring device is a wearable device. In embodiments, the blink reflex monitoring device can be worn by a user and/or patient. In embodiments, the blink reflex monitoring device is secured to a user's and/or patient's head via one or more straps. In embodiments, the blink reflex monitoring device is portable.

In embodiments, the blink reflex monitoring device may be configured to be disposed on a mount or stand to easily allow for consecutive uses by multiple users. In embodiments, securing the blink reflex monitoring device to a tripod or stand allows for multiple users or patients to each quickly and efficiently use the device without having to constantly remove the device from a user's or patient's head.

In embodiments, the blink reflex monitoring device has or comprises:
a front end, a back end, a first plane extending through the front end and the back end,
a second plane intersecting the first plane between the front end and the back end, the back end configured to receive a portion of a face of a user and disposed opposite the front end; and
a strap coupled to the blink reflex monitoring device and having a first position and a second position,
   wherein in the first position the strap extends away from the blink reflex monitoring device along the first plane end and is configured to secure the blink reflex monitoring device to a head of a user and in the second position the strap extends below the blink reflex monitoring device along the second plane and is configured to support the blink reflex monitoring device above a surface.

In embodiments, the blink reflex monitoring device has or comprises:
a front end and a back end, the back end configured to receive a portion of a face of a user and disposed opposite the front end;
at least one strap coupled to the blink reflex monitoring device proximate the back end, the at least one strap configured to be secured around a head of a user; and
an air compression system including a source of compressed air and a valve, the air compression system disposed within the blink reflex monitoring device and the valve configured to output compressed air from the back end at a pressure of about 3 PSI to about 60 PSI.

In embodiments, the air compression system disposed within the blink reflex monitoring device and the valve configured to output compressed air from the back end at a pressure of about 30 PSI to about 50 PSI.

In embodiments, the blink reflex is not measured with an electromyography (EMG) or electrooculography (EOG) device. In embodiments, the blink reflex is measured with a high-speed camera. In embodiments, the blink reflex is not measured with an EMG sensor or EOG electrode.

In embodiments, the measuring of a blink reflex, comprises:
providing a blink reflex monitoring device to a user, the blink reflex monitoring device having an air compression system including a compressed air source and a valve;
outputting a first burst of compressed air from the compressed air source through the valve at a first pressure;
detecting a presence or an absence of a blink in response to the first burst of compressed air source at the first pressure;
if the blink is present, then measuring a duration between the output of the first burst of compressed air at the first pressure and the blink to determine the blink reflex; and
if the blink is absent, then outputting a second burst of compressed air from the compressed air source at a second pressure, the second pressure being greater than the first pressure and measuring a duration between the output of the second burst of compressed air at the second pressure and the blink to determine the blink reflex.

In embodiments, the method of the present disclosure can detect a blink reflex. In embodiments, the method of the present disclosure can detect a blink reflex and distinguish the blink reflex from a false-positive or a false-negative blink. In embodiments, the method of the present disclosure can detect a blink reflex and distinguish the blink reflex from an abnormal blink. In embodiments, the abnormal blink includes double blink, excessive blink, blinking during micro-sleep, and the like. Abnormal blink may occur when one or both eyes transition from the open state to the close state and begin returning to the open state at rate that is substantially slower than that associated with a normal blink. Such abnormal blink may be an indication of the user or patient experiencing fatigue and/or may occur over a period of that that is substantially longer than the normal blink reflex (e.g., without limitation, 5 times longer, 10 times longer, or 20 times longer). In embodiments, the method of the present disclosure omits or flags the abnormal blink from the final results generated.

In embodiments, the one or more blink-associated parameters are selected from:
(a) latency, optionally in milliseconds, comprising a time differential between stimulation and eyelid movement, e.g., one or more of upper eyelid or lower eyelid;
(b) differential latency, optionally in milliseconds, comprising a time differential between the start of ipsilateral eye movement and the start of contralateral eye movement;
(c) delta 30, comprising a time difference between ipsilateral eye and contralateral eye movement;
(d) eyelid excursion, optionally in pixels, comprising a distance traveled by the eyelid from the tonic lid position to closed position;
(e) initial lid velocity, optionally in pixels/msec, comprising an average eyelid speed following start of eyelid movement, e.g. the first about 5 frames, the first about 7 frames, or the first about 10 frames;
(f) time to close, optionally in log scale, comprising a time for lid to travel from tonic lid position to the closed position;
(g) time to open, optionally in log scale, comprising a time for lid to travel from closed position back to tonic lid position;
(h) time under threshold, optionally in log scale, comprising a time that the eyelid spends below the threshold position;
(i) number of oscillations, comprising cycles of up and down upper eyelid movement after a stimulated blink;
(j) total blink time, optionally in log scale, comprising a time from start of eyelid movement until it returns to its tonic lid position;
(k) number of blinks;
(l) blink rate, optionally per minute;
(m) area under curve, optionally in pixels;
(n) maximum closing velocity, optionally in milliseconds, comprising a maximum velocity during eyelid closure;
(o) maximum opening velocity, optionally in milliseconds, comprising a maximum velocity during eyelid opening; and
(p) time to first oscillation, optionally in log scale, comprising the elapsed time between the eyelid returning within threshold of a tonic position for a stimulated blink and the onset of the next unstimulated blink.

In embodiments, in comparison of the patient before receiving the ADHD treatment to after receiving the ADHD treatment
latency decreases;
differential latency decreases;
delta 30 decreases;
eyelid excursion decreases;
initial lid velocity decreases;
time to close decreases;
time to open decreases;
time under threshold decreases;
number of oscillations decreases;
total blink time decreases;
number of blinks decreases;
blink rate decreases;
area under curve decreases;
maximum closing velocity increases;
maximum opening velocity decreases; and/or
time to first oscillation increases.

In embodiments, by “increase” or “decreases”, e.g. in comparison of the one or more blink-associated parameters from a patient before receiving the ADHD treatment to after receiving the ADHD treatment, the disclosure refers to values that change over time or relative to each other. In embodiments “increase” is synonymous with “is more than” and/or reflects a non-static value that has changed. In embodiments “decrease” is synonymous with “is less than” and/or reflects a non-static value that has changed. In embodiments, assessment of whether a value “increases” or “is more than” or “decreases” or “is less than”, e.g. in comparison of the one or more blink-associated parameters from a patient before receiving the ADHD treatment to after receiving the ADHD treatment, involves comparing a measured value at two different time points. The different time points, in embodiments, relate to a before and after period of taking an ADHD medication.

In embodiments, in comparison of the patient before receiving the ADHD treatment to after receiving the ADHD treatment latency decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment differential latency decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment delta 30 decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment eyelid excursion decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment initial lid velocity decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment time to close decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment time to open decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, or by about or at least about 21%, or by about or at least about 22%, or by about or at least about 23%, or by about or at least about 24%, or by about or at least about 25%.

In embodiments, in comparison of the patient before receiving the ADHD treatment to after receiving the ADHD treatment time under threshold decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment number of oscillations decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment number of blinks decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment blink rate decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of the patient before receiving the ADHD treatment to after receiving the ADHD treatment area under curve decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of the patient before receiving the ADHD treatment to after receiving the ADHD treatment maximum closing velocity increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of the patient before receiving the ADHD treatment to after receiving the ADHD treatment maximum opening velocity decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of the patient before receiving the ADHD treatment to after receiving the ADHD treatment time to first oscillation increases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison to a patient who is not afflicted with ADHD, the patient who is afflicted with ADHD will exhibit one or more of the following changes to blink reflex response parameters:
latency decreases;
differential latency decreases;
delta 30 decreases;
eyelid excursion increases;
initial lid velocity increases;
time to close increases;
time to open increases;
time under threshold increases;
number of oscillations increases;
total blink time increases;
number of blinks increases;
blink rate increases;
area under curve increases;
maximum closing velocity increases;
maximum opening velocity increases; and/or
time to first oscillation increases.

In embodiments, by “increase” or “decreases”, e.g. in comparison of a patient who is not afflicted with ADHD with a patient who is afflicted with ADHD, the one or more blink-associated parameters do not change but one or more are static values that are compared to each other. In embodiments “increase” is synonymous with “is more than”. In embodiments “decrease” is synonymous with “is less than”. In embodiments, assessment of whether a value “increases” or “is more than” or “decreases” or “is less than”, e.g. in comparison of a patient who is not afflicted with ADHD with a patient who is afflicted with ADHD, involves comparing a measured value, e.g. from a patient who is afflicted with ADHD, with a references standard, e.g. from a patient or population of patients who are not afflicted with ADHD. Accordingly, in embodiments, the one or more blink-associated parameters of a patient or population of patients who is not afflicted with ADHD are a reference value.

In embodiments, in comparison of a patient who is not afflicted with ADHD with a patient who is afflicted with ADHD latency decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 2.1%, or by about or at least about 2.2%, or by about or at least about 2.3%, or by about or at least about 2.4%, or by about or at least about 2.5%, or by about or at least about 3.0%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of a patient who is not afflicted with ADHD with a patient who is afflicted with ADHD differential latency decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of a patient who is not afflicted with ADHD with a patient who is afflicted with ADHD delta 30 decreases by about or at least about 0.5%, or by about or at least about 1%, or by about or at least about 1.5%, or by about or at least about 2%, or by about or at least about 2.5%, or by about or at least about 3%, or by about or at least about 3.5%, by about or at least about 4.0%, or by about or at least about 4.5%, or by about or at least about 5.0%, or by about or at least about 5.5%, or by about or at least about 6.0%, or by about or at least about 6.5%, or by about or at least about 7.0%, by about or at least about 7.5%, or by about or at least about 8.0%, or by about or at least about 8.5%, or by about or at least about 9.0%, or by about or at least about 9.5%, or by about or at least about 10.0%.

In embodiments, in comparison of a patient who is not afflicted with ADHD with a patient who is afflicted with ADHD eyelid excursion decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of a patient who is not afflicted with ADHD with a patient who is afflicted with ADHD initial lid velocity decreases or increases by about or at least about 0.5%, or by about or at least about 1%, or by about or at least about 1.5%, or by about or at least about 2%, or by about or at least about 2.5%, or by about or at least about 3%, or by about or at least about 3.5%, or by about or at least about 4%, or by about or at least about 4.5%, or by about or at least about 5%, or by about or at least about 5.5%, or by about or at least about 6%, or by about or at least about 6.5%, or by about or at least about 7%, or by about or at least about 7.5%, or by about or at least about 8%, or by about or at least about 8.5%, or by about or at least about 9%, or by about or at least about 9.5%, or by about or at least about 10%, or by about or at least about 10.5%, or by about or at least about 11%, or by about or at least about 11.5%, or by about or at least about 12%, or by about or at least about 12.5%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, by about or at least about 21%, or by about or at least about 22%, or by about or at least about 23%, or by about or at least about 24%, or by about or at least about 25%.

In embodiments, in comparison of a patient who is not afflicted with ADHD with a patient who is afflicted with ADHD time to close decreases or increases by about or at least about 0.1%, or by about or at least about 0.2%, or by about or at least about 0.3%, or by about or at least about 0.4%, or by about or at least about 0.5%, or by about or at least about 0.6%, or by about or at least about 0.7%, or by about or at least about 0.8%, or by about or at least about 0.9%, or by about or at least about 1.00%, or by about or at least about 1.1%, or by about or at least about 1.2%, or by about or at least about 1.3%, or by about or at least about 1.4%, or by about or at least about 1.5%, or by about or at least about 1.6%, or by about or at least about 1.7%, or by about or at least about 1.8%, or by about or at least about 1.9%, or by about or at least about 2.0%, or by about or at least about 3%, or by about or at least about 4%, or by about or at least about 5%, or by about or at least about 6%, or by about or at least about 7%, or by about or at least about 8%, or by about or at least about 9%, or by about or at least about 10%.

In embodiments, in comparison of a patient who is not afflicted with ADHD with a patient who is afflicted with ADHD time to open decrease or increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%, or by about or at least about 55%, or by about or at least about 60%, or by about or at least about 65%, or by about or at least about 70%, or by about or at least about 75%, or by about or at least about 80%, or by about or at least about 85%, or by about or at least about 90%, or by about or at least about 95%, or by about or at least about 100%.

In embodiments, in comparison of a patient who is not afflicted with ADHD with a patient who is afflicted with ADHD time under threshold decreases or increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%.

In embodiments, in comparison of a patient who is not afflicted with ADHD with a patient who is afflicted with ADHD number of oscillations decreases or increases by about or at least about 5%, or by about or at least about 10%, or by about or at least about 15%, or by about or at least about 20%, or by about or at least about 25%.

In embodiments, in comparison of a patient who is not afflicted with ADHD with a patient who is afflicted with ADHD number of blinks decreases or increases by about or at least about 5%, or by about or at least about 10%, or by about or at least about 15%, or by about or at least about 20%, or by about or at least about 25%.

In embodiments, in comparison of a patient who is not afflicted with ADHD with a patient who is afflicted with ADHD blink rate decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, or by about or at least about 21%, or by about or at least about 22%, or by about or at least about 23%, or by about or at least about 24%, or by about or at least about 25%.

In embodiments, in comparison of a patient who is not afflicted with ADHD with a patient who is afflicted with ADHD area under curve decrease or increases by about or at least about 5%, or by about or at least about 10%, or by about or at least about 15%, or by about or at least about 20%, or by about or at least about 25%, or by about or at least about 30%, or by about or at least about 35%, or by about or at least about 40%, or by about or at least about 45%, or by about or at least about 50%, or by about or at least about 55%, or by about or at least about 60%, or by about or at least about 65%, or by about or at least about 70%, or by about or at least about 75%, or by about or at least about 80%, or by about or at least about 85%, or by about or at least about 90%, or by about or at least about 95%, or by about or at least about 100%.

In embodiments, in comparison of a patient who is not afflicted with ADHD with a patient who is afflicted with ADHD maximum closing velocity decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, or by about or at least about 21%, or by about or at least about 22%, or by about or at least about 23%, or by about or at least about 24%, or by about or at least about 25%.

In embodiments, in comparison of a patient who is not afflicted with ADHD with a patient who is afflicted with ADHD maximum opening velocity decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, or by about or at least about 21%, or by about or at least about 22%, or by about or at least about 23%, or by about or at least about 24%, or by about or at least about 25%.

In embodiments, in comparison of a patient who is not afflicted with ADHD with a patient who is afflicted with ADHD time to first oscillation decreases or increases by about or at least about 1%, or by about or at least about 2%, or by about or at least about 3%, or by about or at least about 4%, or by about or at least about 5%, or by about or at least about 6%, or by about or at least about 7%, or by about or at least about 8%, or by about or at least about 9%, or by about or at least about 10%, or by about or at least about 11%, or by about or at least about 12%, or by about or at least about 13%, or by about or at least about 14%, or by about or at least about 15%, or by about or at least about 16%, or by about or at least about 17%, or by about or at least about 18%, or by about or at least about 19%, or by about or at least about 20%, or by about or at least about 21%, or by about or at least about 22%, or by about or at least about 23%, or by about or at least about 24%, or by about or at least about 25%.

In embodiments, the ADHD treatment is medication or nonmedication. In embodiments, the medication treatment is or comprises a stimulant medication, optionally methylphenidate, amphetamine, dextroamphetamine, and/or mixture thereof. In embodiments, the medication treatment is or comprises a nonstimulant medication, optionally atomoxetine.

In embodiments, the ADHD treatment is or comprises one or more of dextroamphetamine, levoamphetamine, lisdexamfetamine, methamphetamine, ADDERALL (amphetamine and dextroamphetamine mixed salts), ADDERALL XR (amphetamine and dextroamphetamine mixed salts), DEXEDRINE (dextroamphetamine sulfate), PROCENTRA (dextroamphetamine sulfate), DEXTROSTAT (dextroamphetamine sulfate), RITALIN (methylphenidate hydrochloride), CONCERTA (methylphenidate extended release), VYVANSE (lisdexamfetamine dimesylate), FOCALIN (dexmethylphenidate hydrochloride), and STRATTERA (atomoxetine hydrochloride), or variants or derivatives thereof.

In embodiments, the ADHD treatment is or comprises a methylphenidate. In embodiments, the ADHD treatment is or comprises a nonstimulant therapy.

In embodiments, the nonmedication treatment is by a brain-computer interface-based software.

In embodiments, the ADHD is diagnosed by Diagnostic and Statistical Manual of Mental Disorders (DSM-5) criteria. In embodiments, the ADHD is diagnosed by International Statistical Classification of Diseases and Related Health Problems (ICD-10) criteria.

In embodiments, the ADHD disorder is diagnosed or diagnosable by one or more of ADHD Rating Scale, Vanderbilt ADHD Diagnostic Rating Scale (VADRS), NICHQ Vanderbilt Assessment Scales, Childhood Attention Problems Scale, Conners' Rating Scales, Conners 3^{rd} Edition: Short version, Behavior Assessment System for Children (BASC), BASC Monitor Rating Scale, Child Behavior Checklist/Teacher Report Form, ADHD Comprehensive Teacher's Rating Scale (ACTeRS): Boys' and girls' form, Childhood Attention Problems Scale, Disruptive Behavior Rating Scale, Swanson, Nolan and Pelham (SNAP) scale, Adult ADHD Self-Report Scale Symptom Checklist Version 1.1 (Adult ASRS), Conners Adult ADHD Rating Scales (CAARS), Wender Utah Rating Scale, Child Behavior Checklist (CBCL), and Brown Attention Deficit Disorder Scales (BADDS).

In embodiments, the ADHD comprises one or more symptoms selected from being easily distracted, forgetful, daydreaming, disorganization, poor concentration, difficulty completing tasks, excessive fidgetiness and restlessness, hyperactivity, difficulty waiting and remaining seated, immature behavior, and/or destructive behavior.

In embodiments, the patient is experiencing symptoms associated with combined type ADHD. In embodiments, the patient is experiencing symptoms associated with predominantly inattentive type ADHD. In embodiments, the patient is experiencing symptoms associated with predominantly hyperactive-impulsive type ADHD.

In embodiments, common signs and symptoms of ADHD include, without limitation, inattention, hyperactivity, disruptive behavior, and impulsivity. In embodiments, hyperactivity is or comprises restlessness. In embodiments, other common signs and symptoms of ADHD include academic difficulties and problems with relationships. In embodiments, such symptoms can be difficult to define, as it is hard to draw a line at where normal levels of inattention, hyperactivity, and impulsivity end and significant levels requiring interventions begin. In embodiments, symptoms of ADHD must be present for six months or more to a degree that is much greater than others of the same age. In embodiments, the patients under the age of 17 require at least six symptoms of either inattention or hyperactivity/impulsivity. In embodiments, the patients aged 17 or older require at least five symptoms of either inattention or hyperactivity/impulsivity. In embodiments, several symptoms must have been present before the age of 12. In embodiments, the symptoms must interfere with or reduce quality of functioning in at least two settings (e.g., social, school, work, or home). In embodiments, the method of the present disclosure provides an effective diagnostic and/or treatment selection for ADHD by utilizing multiple blink reflex parameters.

In embodiments, symptoms for inattentive type ADHD are listed in DSM-5 as frequently overlooks details or making careless mistakes; often has difficulty maintaining focus on one task or play activity; often appears not to be listening when spoken to, including when there is no obvious distraction; frequently does not finish following instructions, failing to complete tasks; Often struggles to organize tasks and activities, to meet deadlines, and to keep belongings in order; Is frequently reluctant to engage in tasks which require sustained attention; frequently loses items, including those required for tasks; is frequently easily distracted by irrelevant things, including thoughts in adults and teenagers; and often forgets daily activities, or is forgetful while completing them.

In embodiments, symptoms for hyperactive-impulsive type ADHD are listed in DSM-5 as is often fidgeting or squirming in seat; frequently has trouble sitting still during dinner, homework, at work, etc.; frequently runs around in inappropriate situations-in adults and teenagers, this may be present as restlessness; often cannot quietly engage in leisure activities or play; Frequently seems to be in constant motion, or uncomfortable when not in motion; often talks too much; Often answers a question before it is finished, or finishes people's sentences; often struggles to wait his or her turn, including waiting in lines; and frequently interrupts or intrudes, including into others' conversations or activities, or by using people's things without asking.

In embodiments, symptom(s) for combined type ADHD is listed in DSM-5 as meet the criteria for both inattentive and hyperactive-impulsive ADHD.

In embodiments, the method of the present disclosure detects, indirectly or directly, a reduction of the total score of an ADHD rating scale in patient after treatment.

In embodiments, the ADHD rating scale is selected from various versions of the Conners Rating Scales, the SNAP scale, the SKAMP scale, the SWAN scale, the ADHD RS-IV scale, the VADTRS scale, the VADPRS scale, the ADHD-SHS scale, the ADDES scale, the ACTers scale, the BADDS scale, the AISRS scale, and the ADHD RS adult, and the like.

In embodiments, there is provided methods of ameliorating one or more symptoms of ADHD and/or selection of agents for ameliorating one or more symptoms of ADHD and/or monitoring if agents are having efficacy in ameliorating one or more symptoms of ADHD. In embodiments, ameliorating one or more symptoms of ADHD includes lessening or improvement of one or more symptoms of ADHD as compared to without treatment. In embodiments, ameliorating one or more symptoms of ADHD includes shortening or reduction in duration of a symptom. For example, in embodiments, one or more symptoms of ADHD is ameliorated by at least about 10%, or at least about 20%, or at least about 30%, or at least about 40%, or at least about 50%, or at least about 60%, or at least about 70% in the individual as compared to the level before treatment.

In embodiments, there is provided methods of controlling ADHD symptoms and/or selection of agents for controlling ADHD symptoms and/or monitoring if agents are having efficacy controlling ADHD symptoms. In embodiments, controlling ADHD symptoms includes maintaining or reducing severity or duration of one or more symptoms of ADHD or frequency of symptoms (e.g., without limitation, frequency of experiencing impulsivity, hyperactivity, and/or inattention) in an individual (as compared to the level before treatment). For example, in embodiments, a duration or severity of one or more symptoms of ADHD is reduced by at least about 10%, or at least about 20%, or at least about 30%, or at least about 40%, or at least about 50%, or at least about 60%, or at least about 70% in the individual as compared to the level before treatment.

In embodiments, there is provided methods of delaying the development of one or more symptoms of ADHD and/or selection of agents having efficacy in delaying the development of one or more symptoms of ADHD and/or monitoring if agents are having efficacy in delaying the development of one or more symptoms of ADHD. In embodiments, delaying the development of one or more symptoms of ADHD includes hindering, slowing, stabilizing, and/or postponing progression of the symptoms (e.g., without limitation, symptoms associated with impulsivity, hyperactivity, and/or inattention). This delay can be of varying lengths of time, depending on the severity and or types of ADHD (e.g., without limitation, predominantly inattentive, predominantly hyperactive-impulsive, or combined type) and/or individuals being treated.

In embodiments, the present disclosure relates to methods involving, e.g., without limitation, selecting or monitoring treatment and/or diagnosing, ADHD and/or the symptoms thereof. In embodiments, ADHD is a disorder characterized by, for example, inattentiveness, over-activity, impulsivity, or a combination thereof. Accordingly, the methods and of the present disclosure are useful for selecting or monitoring treatment and/or diagnosing of inattentiveness, over-activity, impulsivity, or a combination thereof. In embodiments, the ADHD is of any type, including but not limited to, combined type ADHD (ADHD-C), predominantly inattentive type ADHD (ADHD-PI or ADHD-I), and predominantly hyperactive-impulsive type ADHD (ADHD-PH or ADHD-HI). In embodiments, ADHD, predominantly inattentive type presents with symptoms including being easily distracted, forgetful, daydreaming, disorganization, poor concentration, and/or difficulty completing tasks. In embodiments, ADHD, predominantly hyperactive-impulsive type presents with excessive fidgetiness and restlessness, hyperactivity, difficulty waiting and remaining seated, immature behavior, and/or destructive behavior. In embodiments, ADHD, combined type (ADHD-C) presents as a combination of symptoms of the two other subtypes.

In embodiments, the present disclosure provides for is useful for selecting or monitoring treatment and/or diagnosing of ADHD and depression and/or anxiety in the same subject.

The efficacy of treating ADHD using the methods of the present disclosure may be assessed by various methods. For example, efficacy may be assessed using ADHD rating scales as described, for example, in Maan et al., (2008) CNS Drugs, 22 (4):275-90, the entire contents of which is hereby incorporated by reference. Illustrative ADHD scales include, for example, the adult ADHD self-report scale (ASRS), the ADHD Behavior Checklist/ADHD Rating Scale, and the ADHD Investigator Symptom Rating Scale (AISRS).

In embodiments, the present methods are useful in detection, indirectly or directly, of a reduction of the total score of an ADHD rating scale. Examples of ADHD rating scales include, in embodiments, but are not limited to the following scales: various versions of the Conners Rating Scales, the SNAP scale, the SKAMP scale, the SWAN scale, the ADHD RS-IV scale, the VADTRS scale, the VADPRS scale, the ADHD-SHS scale, the ADDES scale, the ACTers scale, the BADDS scale, the AISRS scale and the ADHD RS adult as well as many other similar scales. The raters for each of these scales may be a subject, clinician or investigator, a parent, a teacher, a significant other, or others.

In embodiments, the subject is undergoing treatment with, and/or the present methods determine the efficacy of response to or likelihood of response to one or more ADHD treatments. In embodiments, the ADHD treatment is selected from an amphetamine, methylphenidate (MPH), and a nonstimulant therapy.

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one.

The term “alleviating” means a reduction in the symptoms of ADHD.

The term “treating” means to alleviate (or to eliminate) at least one symptom of ADHD.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

This invention is further illustrated by the following non-limiting examples.

### EXAMPLES

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1

### Clinical Assessment of Blink Reflex Response Differences in Subjects With or Without ADHD and Before and After Pharmacological Intervention

Participants (n=42) were enrolled in a pilot study and segmented into two groups based on ADHD diagnosis, (24 ADHD; 18 non-ADHD control). Groups were statistically evaluated for differences in age, sex, and vitals at baseline (p values indicate no statistically significant differences between groups). A groupwise comparison of the demographics and clinical characteristics is shown below in TABLE 9.

TABLE 1
Groupwise demographics and clinical characteristics at baseline; *fisher's
exact test; **chi-squared test; p-value t-test = independent sample
t-test; p-value MW-u = non-parametric Mann Whitney U test.

| | | | p-value | p-value |
|---|---|---|---|---|
| | ADHD | Control | t-test | MW-u |
| Sample size, n | 24 | 18 | | |
| Age, years | 20.87 ± 1.25 | 20.86 ± 1.13 | 0.982 | 0.939 |
| Sex | | | | >0.999* |
| Male | 21 | 16 | | |
| Female | 3 | 2 | | |
| Race | | | | 0.269** |
| African American | 1 | 3 | | |
| Asian | 1 | 0 | | |
| Bi-racial | 2 | 0 | | |
| Black | 2 | 0 | | |
| Caucasian | 17 | 15 | | |
| Mixed | 1 | 0 | | |
| Hours of Sleep (hr.) | 6.00 ± 1.57 | 5.94 ± 0.88 | 0.890 | 0.807 |
| Height (in.) | 71.43 ± 2.10 | 71.83 ± 0.937 | 0.524 | 0.874 |
| Weight (lbs.) | 194.64 ± 52.86 | 197.08 ± 39.37 | 0.896 | 0.504 |
| Systolic BP (mmHg) | 125.83 ± 19.99 | 116.17 ± 9.81 | 0.067 | 0.181 |
| Diastolic BP (mmHg) | 73.92 ± 10.67 | 72.22 ± 8.28 | 0.579 | 0.575 |
| Heart Rate (bpm) | 78.12 ± 12.22 | 73.17 ± 10.84 | 0.180 | 0.127 |
| Urine Specific Gravity | 1.0172 ± 0.0072 | 1.017 ± 0.006 | 0.600 | 0.669 |

Participants were stratified into two groups: one group with ADHD diagnosis and prescribed ADHD medication (ADHD Medication Group; n=24), and one group that was not diagnosed with ADHD and does not take ADHD medication (Controls; n=18), where clinical assessment for blink reflex response was measured at 2 timepoints. Timepoints were approximately 1 hr. apart, where baseline measurements were gathered before the ADHD cohort took their prescribed medication and 1 hour after taking medication (on the same day). The ADHD group took their medication with a glass of water, whereas control group drank an equivalent volume of water. The 1 hr. timepoint was selected because typically prescribed amphetamine-based ADHD medications require only 30-45 min for effect.

Independent sample t-test and non-parametric Mann Whitney U tests were used to compare continuous variables and Chi squared (or Fishers Exact) tests were used to compare categorical or binary variables. Evaluation determined that the no differences were observed between the ADHD and control groups across demographics and general clinical parameters.

TABLE 2
Groupwise clinical characteristics at second assessment.

| | ADHD | | p-value | p-value |
|---|---|---|---|---|
| | Post-Med | Control | t-test | MW-u |
| Sample size, n | 24 | 17 | | |
| Systolic Blood Pressure | 128.29 ± 14.31 | 118.61 ± 8.72 | 0.015 | 0.015 |
| Diastolic Blood Pressure | 77.92 ± 12.37 | 71.39 ± 9.27 | 0.068 | 0.065 |
| Heart Rate (bpm) | 76.96 ± 13.22 | 67.61 ± 9.76 | 0.016 | 0.016 |
| Urine Specific Gravity | 1.011 ± 0.007 | 1.013 ± 0.006 | 0.381 | 0.317 |

Groupwise clinical characteristics (e.g., as summarized in TABLE 10) indicated a statistically significant difference in systolic BP and heart rate was observed between subjects with ADHD after taking medication as compared to controls. Urine specific gravity and heart rate were determined to be equivalent, indicating that the groups were similarly hydrated.

TABLE 3
Groupwise blink reflex parameters at baseline.

| | ADHD | Control | p-value | p-value |
|---|---|---|---|---|
| | (n = 24) | (n = 18) | t-test | MW-u |
| Latency (ms) | 45.98 ± 7.18 | 46.81 ± 6.36 | 0.700 | 0.381 |
| Differential Latency (ms) | 4.84 ± 2.04 | 5.47 ± 1.66 | 0.294 | 0.195 |
| Delta 30 (ms) | 5.24 ± 2.24 | 6.13 ± 2.52 | 0.233 | 0.208 |
| Initial Velocity (pixels/ms) | 4.38 ± 0.68 | 3.88 ± 0.81 | 0.037 | 0.038 |
| Time to Close (ms) | 30.69 ± 2.62 | 30.30 ± 3.77 | 0.690 | 0.629 |
| Time to Open (ms) | 312.23 ± 213.02 | 198.79 ± 160.18 | 0.066 | 0.015 |
| Time under 20 (ms) | 130.99 ± 79.96 | 98.54 ± 79.91 | 0.200 | 0.057 |
| Oscillations (no.) | 10.35 ± 4.66 | 9.15 ± 4.03 | 0.384 | 0.423 |
| Blinks (no.) | 3.93 ± 1.66 | 3.75 ± 1.34 | 0.702 | 0.819 |
| Blink Rate (per min) | 63.68 ± 27.84 | 60.62 ± 20.47 | 0.696 | 0.879 |
| Area Under Curve (pixels) | 31282 ± 20319 | 18935 ± 13146 | 0.030 | 0.013 |
| Max Closing Velocity | 8.09 ± 1.88 | 7.12 ± 2.10 | 0.126 | 0.093 |
| (pixels/ms) | | | | |
| Max Opening Velocity | 2.600 ± 1.07 | 2.21 ± 0.70 | 0.187 | 0.381 |
| (pixels/ms) | | | | |
| Excursion (pixels) | 116.96 ± 17.50 | 105.30 ± 19.62 | 0.049 | 0.071 |

While no differences in clinical characteristics were observed between the two groups at baseline, statistically significant differences were observed in baseline blink reflex parameters, with the ADHD group having significantly increased initial velocity, increased time to open, and an increased area under curve in comparison to non-ADHD controls. The ADHD medication group exhibited higher excursions as determined by a parametric t-test, but this significance did not carry when a non-parametric Mann Whitney U test was performed, which is the more appropriate test for this comparison.

TABLE 4
Groupwise blink reflex parameters at second assessment.

| | ADHD | | | |
|---|---|---|---|---|
| | Post-Med | Control | p-value | p-value |
| | (n = 24) | (n = 18) | t-test | MW-u |
| Latency (ms) | 45.20 ± 5.19 | 47.02 ± 6.79 | 0.329 | 0.423 |
| Differential Latency (ms) | 4.48 ± 1.84 | 5.99 ± 2.56 | 0.032 | 0.038 |
| Delta 30 (ms) | 4.89 ± 1.90 | 7.91 ± 3.28 | 0.002 | 0.002 |
| Initial Velocity (pixels/ms) | 4.41 ± 0.72 | 3.72 ± 0.87 | 0.007 | 0.004 |
| Time to Close (ms) | 29.90 ± 3.48 | 29.70 ± 2.97 | 0.844 | >0.999 |
| Time to Open (ms) | 258.96 ± 186.26 | 209.19 ± 199.82 | 0.411 | 0.047 |
| Time under 20 (ms) | 120.68 ± 72.37 | 100.53 ± 72.43 | 0.377 | 0.155 |
| Oscillations (no.) | 9.58 ± 5.22 | 8.89 ± 4.56 | 0.658 | 0.859 |
| Blinks (no.) | 3.51 ± 1.71 | 3.45 ± 1.87 | 0.907 | 0.900 |
| Blink Rate (per min) | 55.37 ± 24.33 | 54.63 ± 28.64 | 0.929 | 0.899 |
| Area Under Curve (pixels) | 26577 ± 19330 | 17962 ± 18903 | 0.157 | 0.007 |
| Max Closing Velocity | 8.26 ± 1.48 | 6.13 ± 2.25 | <0.001 | <0.001 |
| (pixels/ms) | | | | |
| Max Opening Velocity | 2.39 ± 0.80 | 2.20 ± 0.92 | 0.490 | 0.751 |
| (pixels/ms) | | | | |
| Excursion (pixels) | 117.16 ± 20.39 | 99.07 ± 22.84 | 0.010 | 0.015 |

At the second assessment timepoint, statistically significant differences were observed in systolic BP and heart rate between ADHD medication group and non-ADHD controls. This was to be anticipated as amphetamine-based ADHD medications are known to increase heart rate and blood pressure. In addition, statistically significant differences were observed in Delta 30 (time difference between ipsilateral eye and contralateral eye movement), initial velocity, area under curve, max closing velocity, and excursion (total pixelate distance traveled) in the ADHD medication group in comparison to non-ADHD controls (e.g., as shown in TABLE 4).

TABLE 5
Comparison in group differences of blink reflex parameters
between baseline and second assessment.

| | ADHD | Control |
|---|---|---|
| | (n = 24) | (n = 18) |
| diff_latency | −0.78 ± 7.07 | 0.21 ± 4.37 |
| diff_differenciallatency | −0.37 ± 2.61 | 0.52 ± 2.01 |
| diff_delta30 | −0.35 ± 2.13 | 1.79 ± 2.72 |
| diff_initialvelocity | 0.03 ± 0.51 | −0.17 ± 0.49 |
| diff_timetoclose | −0.79 ± 2.87 | −0.59 ± 3.46 |
| diff_timetoopen | −53.26 ± 216.61 | 10.41 ± 85.52 |
| diff_timeunder20 | −10.31 ± 63.24 | 1.99 ± 40.73 |
| diff_oscillation | −0.77 ± 2.86 | −0.25 ± 2.61 |
| diff_blinks | −0.42 ± 1.63 | −0.30 ± 1.88 |
| diff_blinkrate | −8.32 ± 25.79 | −5.99 ± 27.78 |
| diff_areaundercurve | −4704.81 ± 22068.63 | −972.92 ± 10931.53 |
| diff_maxclosingvelocity | 0.17 ± 1.80 | −1.00 ± 3.24 |
| diff_maxopeningvelocity | −0.21 ± 0.91 | −0.01 ± 0.72 |
| diff_excursions | 0.20 ± 16.69 | −6.23 ± 14.86 |

TABLE 5 summarizes the mean differences in each blink reflex parameter within each group between timepoints, differences are reported as mean change±S.D. from baseline to second assessment. It was observed that a larger mean difference in the controls in Delta 30 was driving the significant results at the post-medication assessment.

To compare the changes in blink parameters from baseline to second assessment, three mixed model approaches were implemented.

Model 1 included the main effects of group and timepoint, and their interaction. This was performed to determine if there are any true differences in the blink reflex response parameters that can be used to accurately discriminate between ADHD status—i.e., be used to diagnose ADHD.

Model 2 included the binary effect of medication in the model. This was performed to determine if the blink reflex response parameters can accurately indicate if a patient is undergoing ADHD medication—i.e., detect adherence to a medication.

Model 3 was used to determine if the class of medication AMP (Amphetamine salts, Adderall, Vyvanse), ATX (Atomoxetine based, non-stimulant), or MPH (methylphenidate) had any effect on the blink reflex response parameters (e.g., test if the parameters can be used to prompt a change in treatment).

All models used an identity covariance structure and determined the degrees of “random effects” for each participant. The Type III fixed effects and estimates of these fixed effects were interpreted. If any significant fixed effects were detected, then additional models using possible confounders were also compared. The correlation between variables of interest were compared and binary logistic regression was used to determine which, if any, variables and their appropriate interactions were predictive of taking ADHD medications. A p-value of less than 0.05 was considered significant.

### Model 1: Mixed Effects Model for Evaluating Changes in Blink Parameters for Diagnosis of ADHD

Model 1 focused on a comparison performed to determine if the differences in the blink reflex parameters between groups is significant (i.e., ADHD Medication at T1 and T2 are different than Control at T1 and T2), and the degree of the interaction between parameters. If the mean change between groups and timepoints is determined to be significant via this model, then the mean change between timepoints (or groups) is indeed indicative of a true difference (e.g., similar to a paired t-test).

TABLE 6
Model 1 Dimensions.

| | | Number of | Covariance | Number of |
|---|---|---|---|---|
| | | Levels | Structure | Parameters |
| Fixed | Intercept | 1 | | 1 |
| Effects | Group | 2 | | 1 |
| | Timepoint | 2 | | 1 |
| | Group * Timepoint | 4 | | 1 |
| Random | Intercept^{b} | 1 | Variance | 1 |
| Effects | | | Components | |

| | | |
|---|---|---|
| Residual | | 1 |
| Total | 10 | 6 |

In Model 1, Group 1 =ADHD medication and Group 2 =Control. The statistical significance of the blink reflex response parameters are indicated, as summarized in TABLES 7-21.

TABLE 7
Model 1 Latency Test for Fixed Effects.
Type III Tests of Fixed Effects^{a}

| Source | Numerator df | Denominator df | F | Sig. |
|---|---|---|---|---|
| Intercept | 1 | 80 | 96.695 | <.001 |
| Group | 1 | 80 | .884 | .350 |
| Timepoint | 1 | 80 | .040 | .841 |
| Group * Timepoint | 1 | 80 | .125 | .725 |

^{a}Dependent Variable: Latency.

Latency was not found to be statistically significant between groups (e.g., ADHD versus control) or between timepoints (e.g., ADHD pre-med versus post-med).

TABLE 8
Model 1 Differential Latency Test for Fixed Effects.
Type III Tests of Fixed Effects^{a}

| Source | Numerator df | Denominator df | F | Sig. |
|---|---|---|---|---|
| Intercept | 1 | 80 | 12.024 | <.001 |
| Group | 1 | 80 | 5.662 | .020 |
| Timepoint | 1 | 80 | .030 | .863 |
| Group * Timepoint | 1 | 80 | .973 | .327 |

^{a}Dependent Variable: DifferentialLatency.

Differential Latency was found to be statistically significant between groups (e.g., ADHD versus control), but not between timepoints (e.g., ADHD pre-med versus post-med) or between timepoints within groups (e.g., ADHD pre-med versus ADHD-post-med difference compared against control baseline versus control second assessment).

TABLE 9
Model 1 Delta 30 Test for Fixed Effects.
Type III Tests of Fixed Effects^{a}

| Source | Numerator df | Denominator df | F | Sig. |
|---|---|---|---|---|
| Intercept | 1 | 80 | 11.064 | .001 |
| Group | 1 | 80 | 12.944 | <.001 |
| Timepoint | 1 | 80 | 1.731 | .192 |
| Group * Timepoint | 1 | 80 | 3.840 | .054 |

^{a}Dependent Variable: Delta30.

TABLE 10
Model 1 Delta 30 Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 7.914 | 1.843 | 80 | 4.294 | <.001 | 4.246 | 11.583 |
| [Timepoint = 1] * [Group = 1] | −2.678 | .771 | 80 | −3.475 | <.001 | −4.212 | −1.144 |
| [Timepoint = 2] * [Group = 1] | −3.029 | .771 | 80 | −3.930 | <.001 | −4.562 | −1.495 |
| [Timepoint = 1] * [Group = 2] | −1.785 | .824 | 80 | −2.167 | .033 | −3.425 | −.145 |
| [Timepoint = 2] * [Group = 2] | 0^{b} | 0 | . | . | . | . | . |

^{a}Dependent Variable: Delta30.
^{b}This parameter is set to zero because it is redundant.

Delta 30 (time difference between ipsilateral eye and contralateral eye movement) was found to be statistically significant between groups (e.g., ADHD versus control) and between group timepoints (e.g., ADHD pre-med versus ADHD-post-med difference compared against control baseline versus control second assessment), but not between timepoints within groups (e.g., ADHD pre-med versus post-med).

TABLE 11
Model 1 Initial Velocity Test for Fixed Effects.
Type III Tests of Fixed Effects^{a}

| Source | Numerator df | Denominator df | F | Sig. |
|---|---|---|---|---|
| Intercept | 1 | 80 | 53.415 | <.001 |
| Group | 1 | 80 | 12.601 | <.001 |
| Timepoint | 1 | 80 | .162 | .689 |
| Group * Timepoint | 1 | 80 | .354 | .553 |

^{a}Dependent Variable: InitialVelocity.

Initial velocity was found to be statistically significant between groups (e.g., ADHD versus control), lacking indication of statistical significance between timepoints and group timepoints.

TABLE 12
Model 1 Time to Close Test for Fixed Effects.
Type III Tests of Fixed Effects^{a}

| Source | Numerator df | Denominator df | F | Sig. |
|---|---|---|---|---|
| Intercept | 1 | 80 | 162.634 | <.001 |
| Group | 1 | 80 | .178 | .675 |
| Timepoint | 1 | 80 | .950 | .333 |
| Group * Timepoint | 1 | 80 | .019 | .892 |

^{a}Dependent Variable: TimetoClose.

Time to close was not found to be statistically significant between groups (e.g., ADHD versus control) or between timepoints (e.g., ADHD pre-med versus post-med).

TABLE 13
Model 1 Time to Open Test for Fixed Effects.
Type III Tests of Fixed Effects^{a}

| Source | Numerator df | Denominator df | F | Sig. |
|---|---|---|---|---|
| Intercept | 1 | 80 | 3.003 | .087 |
| Group | 1 | 80 | 3.708 | .058 |
| Timepoint | 1 | 80 | .256 | .615 |
| Group * Timepoint | 1 | 80 | .564 | .455 |

^{a}Dependent Variable: TimetoOpen.

Time to open was not found to be statistically significant between groups (e.g., ADHD versus control) or between timepoints (e.g., ADHD pre-med versus post-med).

TABLE 14
Model 1 Time under 20 Test for Fixed Effects.
Type III Tests of Fixed Effects^{a}

| Source | Numerator df | Denominator df | F | Sig. |
|---|---|---|---|---|
| Intercept | 1 | 80 | 4.044 | .048 |
| Group | 1 | 80 | 2.447 | .122 |
| Timepoint | 1 | 80 | .061 | .805 |
| Group * Timepoint | 1 | 80 | .134 | .716 |

^{a}Dependent Variable: Timeunder20.

Time under 20 was not found to be statistically significant between groups (e.g., ADHD versus control) or between timepoints (e.g., ADHD pre-med versus post-med).

TABLE 15
Model 1 Oscillations Test for Fixed Effects.
Type III Tests of Fixed Effects^{a}

| Source | Numerator df | Denominator df | F | Sig. |
|---|---|---|---|---|
| Intercept | 1 | 80 | 7.608 | .007 |
| Group | 1 | 80 | .844 | .361 |
| Timepoint | 1 | 80 | .245 | .622 |
| Group * Timepoint | 1 | 80 | .063 | .802 |

^{a}Dependent Variable: Oscillations.

The number of oscillations was not found to be statistically significant between groups (e.g., ADHD versus control) or between timepoints (e.g., ADHD pre-med versus post-med).

TABLE 16
Model 1 Blinks Test for Fixed Effects.
Type III Tests of Fixed Effects^{a}

| Source | Numerator df | Denominator df | F | Sig. |
|---|---|---|---|---|
| Intercept | 1 | 80 | 8.997 | .004 |
| Group | 1 | 80 | .116 | .734 |
| Timepoint | 1 | 80 | .976 | .326 |
| Group * Timepoint | 1 | 80 | .026 | .871 |

^{a}Dependent Variable: Blinks.

The number of blinks was not found to be statistically significant between groups (e.g., ADHD versus control) or between timepoints (e.g., ADHD pre-med versus post-med).

TABLE 17
Model 1 Blink Rate Test for Fixed Effects.
Type III Tests of Fixed Effects^{a}

| Source | Numerator df | Denominator df | F | Sig. |
|---|---|---|---|---|
| Intercept | 1 | 80 | 9.683 | .003 |
| Group | 1 | 80 | .113 | .738 |
| Timepoint | 1 | 80 | 1.604 | .209 |
| Group * Timepoint | 1 | 80 | .042 | .837 |

^{a}Dependent Variable: BlinkRate.

The blink rate was not found to be statistically significant between groups (e.g., ADHD versus control) or between timepoints (e.g., ADHD pre-med versus post-med).

TABLE 18
Model 1 Area Under Curve (AUC) Test for Fixed Effects.
Type III Tests of Fixed Effects^{a}

| Source | Numerator df | Denominator df | F | Sig. |
|---|---|---|---|---|
| Intercept | 1 | 80 | 3.068 | .084 |
| Group | 1 | 80 | 6.672 | .012 |
| Timepoint | 1 | 80 | .489 | .486 |
| Group * Timepoint | 1 | 80 | .211 | .647 |

^{a}Dependent Variable: AreaUnderCurve.

AUC was found to be statistically significant between groups (e.g., ADHD versus control), lacking indication of statistical significance between timepoints and group timepoints.

TABLE 19
Model 1 Max Closing Velocity Test for Fixed Effects.
Type III Tests of Fixed Effects^{a}

| Source | Numerator df | Denominator df | F | Sig. |
|---|---|---|---|---|
| Intercept | 1 | 80 | 27.721 | <.001 |
| Group | 1 | 80 | 13.508 | <.001 |
| Timepoint | 1 | 80 | .949 | .333 |
| Group * Timepoint | 1 | 80 | 1.928 | .169 |

^{a}Dependent Variable: MaxClosingVelocity.

Max Closing Velocity was found to be statistically significant between groups (e.g., ADHD versus control), lacking indication of statistical significance between timepoints and group timepoints.

TABLE 20
Model 1 Max Opening Velocity Test for Fixed Effects.
Type III Tests of Fixed Effects^{a}

| Source | Numerator df | Denominator df | F | Sig. |
|---|---|---|---|---|
| Intercept | 1 | 80 | 12.814 | <.001 |
| Group | 1 | 80 | 2.131 | .148 |
| Timepoint | 1 | 80 | .322 | .572 |
| Group * Timepoint | 1 | 80 | .270 | .605 |

^{a}Dependent Variable: MaxOpeningVelocity.

Max Opening Velocity was not found to be statistically significant between groups (e.g., ADHD versus control) or between timepoints (e.g., ADHD pre-med versus post-med).

TABLE 21
Model 1 Excursions Test for Fixed Effects.
Type III Tests of Fixed Effects^{a}

| Source | Numerator df | Denominator df | F | Sig. |
|---|---|---|---|---|
| Intercept | 1 | 80 | 55.592 | <.001 |
| Group | 1 | 80 | 11.376 | .001 |
| Timepoint | 1 | 80 | .468 | .496 |
| Group * Timepoint | 1 | 80 | .532 | .468 |

^{a}Dependent Variable: Excursion.

Excursions (total pixelate distance covered by eyelid movement) was found to be statistically significant between groups (e.g., ADHD versus control), lacking indication of statistical significance between timepoints and group timepoints.

In conclusion, Model 1 indicated that the ADHD Medication group as a whole is significantly different from the non-ADHD control group and the blink reflex response parameters can be reliably used to diagnose ADHD. But, this model appeared to show no significant differences between the pre-med and post-med timepoints within the ADHD group. Delta 30 was the only interaction approaching significance in this respect, but it is observed that the control at baseline is statistically different than controls at second assessment, which is driving the significance in their interaction term. These statistical analyses indicate that the blink response parameters can also be used to determine if a patient is adhering to an ADHD treatment regimen. Larger sample sizes will likely strengthen the value of parameters that lacked statistical significance and are still considered relevant for diagnosis.

### Model 2: ADHD Medication Class as a Predictor of Blink Response Modulation

Model 2 focused on the binary effect of medication instead of Group as a fixed effect and uses time in the repeated structure (e.g., the effect of pre-med to post-med was evaluated).

TABLE 22
Model 2 Dimensions.
Model Dimension^{a}

| | | Number of | Covariance | Number of | Subject | Number of |
|---|---|---|---|---|---|---|
| | | Levels | Structure | Parameters | Variables | Subjects |
| Fixed | Intercept | 1 | | 1 | | |
| Effects | Treatment | 2 | | 1 | | |
| Random | Intercept^{b} | 1 | Variance | 1 | | |
| Effects | | | Components | | | |
| Repeated | Timepoint | 2 | Identity | 1 | PatientID | 42 |
| Effects | | | | | | |

| | | |
|---|---|---|
| Total | 6 | 4 |

TABLE 23
Model 2 Latency Test for Fixed Effects.
Type III Tests of Fixed Effects^{a}

| Source | Numerator df | Denominator df | F | Sig. |
|---|---|---|---|---|
| Intercept | 1 | 82 | 101.818 | <.001 |
| Treatment | 1 | 82 | .771 | .383 |

^{a}Dependent Variable: Latency.

Latency was not found to be statisticaly significant between timepoints (e.g., ADHD pre-med versus post-med).

TABLE 24
Model 2 Differential Latency Test for Fixed Effects.
Type III Tests of Fixed Effects^{a}

| Source | Numerator df | Denominator df | F | Sig. |
|---|---|---|---|---|
| Intercept | 1 | 82 | 11.159 | .001 |
| Treatment | 1 | 82 | 3.272 | .074 |

^{a}Dependent Variable: DifferentialLatency.

Differential was not found to be statisticaly significant between timepoints (e.g., ADHD pre-med versus post-med).

TABLE 25
Model 2 Delta 30 Test for Fixed Effects.
Type III Tests of Fixed Effects^{a}

| Source | Numerator df | Denominator df | F | Sig. |
|---|---|---|---|---|
| Intercept | 1 | 82 | 8.855 | .004 |
| Treatment | 1 | 82 | 5.045 | .027 |

^{a}Dependent Variable: Delta30.

TABLE 26
Model 2 Delta 30 Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 4.886 | 1.930 | 82 | 2.532 | .013 | 1.047 | 8.725 |
| [Treatment = 0] | 1.422 | .633 | 82 | 2.246 | .027 | .163 | 2.681 |
| [Treatment = 1] | 0^{b} | 0 | . | . | . | . | . |

^{a}Dependent Variable: Delta30.
^{b}This parameter is set to zero because it is redundant.

Delta 30 (time difference between ipsilateral eye and contralateral eye movement) was found to be statisticaly significant between treatment group timepoints (e.g., ADHD pre-med versus ADHD-post-med).

TABLE 27
Model 2 Initial Velocity Test for Fixed Effects.
Type III Tests of Fixed Effects^{a}

| Source | Numerator df | Denominator df | F | Sig. |
|---|---|---|---|---|
| Intercept | 1 | 82 | 54.922 | <.001 |
| Treatment | 1 | 82 | 3.948 | .050 |

^{a}Dependent Variable: InitialVelocity.

TABLE 28
Model 2 Initial Velocity Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 4.414 | .585 | 82 | 7.549 | <.001 | 3.251 | 5.577 |
| [Treatment = 0] | −.381 | .192 | 82 | −1.987 | .050 | −.763 | .000 |
| [Treatment = 1] | 0^{b} | 0 | . | . | . | . | . |

^{a}Dependent Variable: InitialVelocity.
^{b}This parameter is set to zero because it is redundant.

Initial velocity was found to be statistically significant between treatment group timepoints (e.g., ADHD pre-med versus ADHD-post-med).

TABLE 29
Model 2 Time to Close Test for Fixed Effects.
Type III Tests of Fixed Effects^{a}

| Source | Numerator df | Denominator df | F | Sig. |
|---|---|---|---|---|
| Intercept | 1 | 82 | 172.036 | <.001 |
| Treatment | 1 | 82 | .233 | .631 |

^{a}Dependent Variable: TimetoClose.

Time to close was not found to be statistically significant between treatment group timepoints (e.g., ADHD pre-med versus ADHD-post-med).

TABLE 30
Model 2 Time to Open Test for Fixed Effects.
Type III Tests of Fixed Effects^{a}

| Source | Numerator df | Denominator df | F | Sig. |
|---|---|---|---|---|
| Intercept | 1 | 2.021 | 3.264 | .211 |
| Treatment | 1 | 82 | .061 | .805 |

^{a}Dependent Variable: TimetoOpen.

Time to open was not found to be statistically significant between treatment group timepoints (e.g., ADHD pre-med versus ADHD-post-med).

TABLE 31
Model 2 Time under 20 Test for Fixed Effects.
Type III Tests of Fixed Effects^{a}

| Source | Numerator df | Denominator df | F | Sig. |
|---|---|---|---|---|
| Intercept | 1 | 2.021 | 4.500 | .167 |
| Treatment | 1 | 82 | .215 | .644 |

^{a}Dependent Variable: Timeunder20.

Time under 20 was not found to be statistically significant between treatment group timepoints (e.g., ADHD pre-med versus ADHD-post-med).

TABLE 32
Model 2 Oscillations Test for Fixed Effects.
Type III Tests of Fixed Effects^{a}

| Source | Numerator df | Denominator df | F | Sig. |
|---|---|---|---|---|
| Intercept | 1 | 82 | 8.183 | .005 |
| Treatment | 1 | 82 | .001 | .980 |

^{a}Dependent Variable: Oscillations.

The number of oscillations was not found to be statistically significant between treatment group timepoints (e.g., ADHD pre-med versus ADHD-post-med).

TABLE 33
Model 2 Blinks Test for Fixed Effects.
Type III Tests of Fixed Effects^{a}

| Source | Numerator df | Denominator df | F | Sig. |
|---|---|---|---|---|
| Intercept | 1 | 82 | 9.366 | .003 |
| Treatment | 1 | 82 | .305 | .582 |

^{a}Dependent Variable: Blinks.

The number of blinks was not found to be statistically significant between treatment group timepoints (e.g., ADHD pre-med versus ADHD-post-med).

TABLE 34
Model 2 Blink Rate Test for Fixed Effects.
Type III Tests of Fixed Effects^{a}

| Source | Numerator df | Denominator df | F | Sig. |
|---|---|---|---|---|
| Intercept | 1 | 2.021 | 9.940 | .086 |
| Treatment | 1 | 82 | .578 | .449 |

^{a}Dependent Variable: BlinkRate.

The blink rate was not found to be statistically significant between treatment group timepoints (e.g., ADHD pre-med versus ADHD-post-med).

TABLE 35
Model 2 Area Under Curve (AUC) Test for Fixed Effects.
Type III Tests of Fixed Effects^{a}

| Source | Numerator df | Denominator df | F | Sig. |
|---|---|---|---|---|
| Intercept | 1 | 2.021 | 3.398 | .205 |
| Treatment | 1 | 82 | .428 | .515 |

^{a}Dependent Variable: AreaUnderCurve.

AUC was not found to be statistically significant between treatment group timepoints (e.g., ADHD pre-med versus ADHD-post-med).

TABLE 36
Model 2 Max Closing Velocity Test for Fixed Effects.
Type III Tests of Fixed Effects^{a}

| Source | Numerator df | Denominator df | F | Sig. |
|---|---|---|---|---|
| Intercept | 1 | 82 | 28.673 | <.001 |
| Treatment | 1 | 82 | 4.681 | .033 |

^{a}Dependent Variable: MaxClosingVelocity.

TABLE 37
Model 2 Max Closing Velocity Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 8.260 | 1.482 | 82 | 5.574 | <.001 | 5.312 | 11.208 |
| [Treatment = 0] | −1.052 | .486 | 82 | −2.164 | .033 | −2.019 | −.085 |
| [Treatment = 1] | 0^{b} | 0 | . | . | . | . | . |

^{a}Dependent Variable: MaxClosingVelocity.
^{b}This parameter is set to zero because it is redundant.

Max Closing Velocity was found to be statistically significant between treatment group timepoints (e.g., ADHD pre-med versus ADHD-post-med).

TABLE 38
Model 2 Max Opening Velocity Test for Fixed Effects.
Type III Tests of Fixed Effects^{a}

| Source | Numerator df | Denominator df | F | Sig. |
|---|---|---|---|---|
| Intercept | 1 | 82 | 13.595 | <.001 |
| Treatment | 1 | 82 | .011 | .917 |

^{a}Dependent Variable: MaxOpeningVelocity.

Max Opening Velocity was not found to be statistically significant between groups (e.g., ADHD versus control) or between timepoints (e.g., ADHD pre-med versus post-med).

TABLE 39
Model 2 Excursions Test for Fixed Effects.
Type III Tests of Fixed Effects^{a}

| Source | Numerator df | Denominator df | F | Sig. |
|---|---|---|---|---|
| Intercept | 1 | 2.021 | 56.871 | .017 |
| Treatment | 1 | 82 | 3.254 | .075 |

^{a}Dependent Variable: Excursion.

Excursions (total pixelate distance covered by eyelid movement) was not found to be statistically significant between groups (e.g., ADHD versus control) or between timepoints (e.g., ADHD pre-med versus post-med).

In conclusion, Model 2 indicated that the ADHD Medication had a significant effect on Delta 30, Initial Velocity, and Max Closing Velocity. However, the controls for these three parameters had the greatest variability. Before using any combination of these parameters to produce a predictive model using logistic regression, we determined the effect of any correlations between these parameters.

TABLE 40
Model 2 Correlation between significant
predictors of ADHD medication efficacy.
Correlations

| | | | | Max |
|---|---|---|---|---|
| | | Delta | Initial | Closing |
| | | 30 | Velocity | Velocity |
| Delta | Pearson Correlation | — | | |
| 30 | N | 84 | | |
| Initial | Pearson Correlation | −0.326** | — | |
| Velocity | Sig. (2-tailed) | 0.002 | | |
| | N | 84 | 84 | |
| Max | Pearson Correlation | −0.322** | 0.660** | — |
| Closing | Sig. (2-tailed) | 0.003 | <0.001 | |
| Velocity | N | 84 | 84 | 84 |

**Correlation is significant at the 0.01 level (2-tailed).

All three variables are significantly correlated, but the correlation between Initial Velocity and Max Closing Velocity was greater than 0.5, violating assumptions used for logistic regression that cannot be overcome using an interaction term, and would thus fail the Hosmer Lemeshow Goodness-of-Fit test. Max closing velocity had a more significant p-value than initial velocity.

TABLE 41
Model 2 Predictive Accuracy.
Variables in the Equation

| | | | | | | | | 95% C.I. for EXP(B) | |
|---|---|---|---|---|---|---|---|---|---|
| | | B | S.E. | Wald | df | Sig. | Exp(B) | Lower | Upper |
| Step 1^{a} | Delta 30 | −0.194 | 0.115 | 2.829 | 1 | 0.093 | 0.824 | 0.657 | 1.033 |
| | Max Closing Velocity | 0.197 | 0.133 | 2.194 | 1 | 0.139 | 1.218 | 0.938 | 1.582 |
| | Constant | −1.368 | 1.354 | 1.020 | 1 | 0.312 | 0.255 | | |

^{a}Variable(s) entered on step 1: Delta 30, Max Closing Velocity.

While this predictive model demonstrated overall poor accuracy (likely due to sample size considerations) it demonstrated some level of predictive power, as summarized in the classification table below (e.g., TABLE 50), and while mostly identical to the null model (but also 71.4% accurate) in predictive potential.

TABLE 42
Model 2 Classification Table.
Classification Table^{a}

| | | | Predicted | | |
|---|---|---|---|---|---|
| | | | Treatment | | Percentage |
| | Observed | | Untreated | Treated | Correct |
| Step 1 | Treatment | Untreated | 57 | 3 | 95.0 |
| | | Treated | 21 | 3 | 12.5 |

| | |
|---|---|
| Overall Percentage | 71.4 |

^{a}The cut value is .500

In conclusion, Model 2 demonstrates that, with respect to ADHD treatment, the combination of two or more blink reflex variables is likely to result in decreased predictive power, but that the blink reflex parameters, in aggregate (e.g., as a set of individual parameters comparisons to control), have usefulness in determining effects of ADHD medication.

### Model 3: Blink Response Modulation as a Predictor of ADHD Medication Class Effectiveness

Model 3 focused on a comparison performed to determine if the differences between medication type can serve as a useful predictor of blink response modulation, and vice versa. Model 3 was performed with an unequal group distribution among treatment type. For example, 20 participants took AMP (Amphetamine salts, Adderall, Vyvanse), 2 participants took ATX (Atomoxetine based, non-stimulant), and 2 participants took MPH (methylphenidate). TABLE 43 below summarizes the treatment key used for the subsequent evaluation (e.g., as shown in TABLES 44-62).

TABLE 43
Model 3 Treatment Key.

| Medication | Med Type number |
|---|---|
| AMP | 1 |
| ATX | 2 |
| MPH | 3 |
| Control | 4 |

TABLE 44
Model 3 Latency Fixed Effects Test.
Type III Tests of Fixed Effects^{a}

| Source | Numerator df | Denominator df | F | Sig. |
|---|---|---|---|---|
| Intercept | 1 | 1250.325 | 371.215 | <.001 |
| Med_type | 3 | 80 | .878 | .456 |

^{a}Dependent Variable: Latency.

Latency was not found to be statisticaly significant between the different ADHD medication types.

TABLE 45
Model 3 Differential Latency Fixed Effects Test.
Type III Tests of Fixed Effects^{a}

| Source | Numerator df | Denominator df | F | Sig. |
|---|---|---|---|---|
| Intercept | 1 | 80 | 157.749 | <.001 |
| Med_type | 3 | 80 | 2.643 | .055 |

^{a}Dependent Variable: DifferentialLatency.

TABLE 46
Model 3 Differential Latency Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 5.731 | 0.337 | 80 | 16.987 | <0.001 | 5.059 | 6.402 |
| [Med_type = 1] | −0.586 | 1.067 | 80 | −0.549 | 0.585 | −2.709 | 1.537 |
| [Med_type = 2] | −2.456 | 1.067 | 80 | −2.302 | 0.024 | −4.579 | −0.333 |
| [Med_type = 3] | −0.980 | 0.465 | 80 | −2.107 | 0.038 | −1.905 | −0.054 |
| [Med_type = 4] | 0^{b} | 0 | . | . | . | . | . |

^{a}Dependent Variable: DifferentialLatency.
^{b}This parameter is set to zero because it is redundant.

Differential Latency was found to be statistically significant between the diferent ADHD medication types, particularly with ATX and MPH.

TABLE 47
Model 3 Delta 30 Fixed Effects Test.
Type III Tests of Fixed Effects^{a}

| Source | Numerator df | Denominator df | F | Sig. |
|---|---|---|---|---|
| Intercept | 1 | .000 | 13.024 | .000 |
| Med_type | 3 | 80 | 5.397 | .002 |

^{a}Dependent Variable: Delta30.

TABLE 48
Model 3 Delta 30 Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 7.022 | 1.396 | .000 | 5.031 | .000 | 4.497 | 9.547 |
| [Med_type = 1] | −1.782 | 1.314 | 80 | −1.356 | .179 | −4.397 | .833 |
| [Med_type = 2] | −4.184 | 1.314 | 80 | −3.184 | .002 | −6.800 | −1.569 |
| [Med_type = 3] | −1.756 | .573 | 80 | −3.066 | .003 | −2.896 | −.616 |
| [Med_type = 4] | 0^{b} | 0 | . | . | . | . | . |

^{a}Dependent Variable: Delta30.
^{b}This parameter is set to zero because it is redundant.

Delta 30 was found to be statistically significant between the different ADHD medication types, particularly with ATX and MPH.

TABLE 49
Model 3 Initial Velocity Fixed Effects Test.
Type III Tests of Fixed Effects^{a}

| Source | Numerator df | Denominator df | F | Sig. |
|---|---|---|---|---|
| Intercept | 1 | .000 | 22.711 | .000 |
| Med_type | 3 | 80 | 4.376 | 007 |

^{a}Dependent Variable: InitialVelocity.

TABLE 50
Model 3 Initial Velocity Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 3.801 | .898 | .000 | 4.230 | 1.000 | −1.505 | 9.106 |
| [Med_type = 1] | .857 | .402 | 80 | 2.131 | .036 | .057 | 1.657 |
| [Med_type = 2] | .532 | .402 | 80 | 1.323 | .190 | −.268 | 1.332 |
| [Med_type = 3] | .578 | .175 | 80 | 3.297 | .001 | .229 | .927 |
| [Med_type = 4] | 0^{b} | 0 | . | . | . | . | . |

^{a}Dependent Variable: InitialVelocity.
^{b}This parameter is set to zero because it is redundant.

Initial Velocity was found to be statistically significant between the different ADHD medication types, particularly with AMP and MPH.

TABLE 51
Model 3 Time to Close Fixed Effects Test.
Type III Tests of Fixed Effects^{a}

| Source | Numerator df | Denominator df | F | Sig. |
|---|---|---|---|---|
| Intercept | 1 | 7841.326 | 252.215 | <.001 |
| Med_type | 3 | 80.000 | .300 | .826 |

^{a}Dependent Variable: TimetoClose.

Time to close was not found to be statistically significant between the ADHD medication types.

TABLE 52
Model 3 Time to Open Fixed Effects Test.
Type III Tests of Fixed Effects^{a}

| Source | Numerator df | Denominator df | F | Sig. |
|---|---|---|---|---|
| Intercept | 1 | 972.721 | 13.852 | <.001 |
| Med_type | 3 | 80.000 | 1.447 | .235 |

^{a}Dependent Variable: TimetoOpen.

Time to open was not found to be statistically significant between the ADHD medication types.

TABLE 53
Model 3 Time under 20 Fixed Effects Test.
Type III Tests of Fixed Effects^{a}

| Source | Numerator df | Denominator df | F | Sig. |
|---|---|---|---|---|
| Intercept | 1 | .000 | 29.575 | .000 |
| Med_type | 3 | 80 | 1.242 | 300 |

^{a}Dependent Variable: Timeunder20.

Time under 20 was not found to be statistically significant between the ADHD medication types.

TABLE 54
Model 3 Oscillations Fixed Effects Test.
Type III Tests of Fixed Effects^{a}

| Source | Numerator df | Denominator df | F | Sig. |
|---|---|---|---|---|
| Intercept | 1 | 80 | 104.872 | <.001 |
| Med_type | 3 | 80 | 1.437 | .238 |

^{a}Dependent Variable: Oscillations.

Oscillations were not found to be statistically significant between the ADHD medication types.

TABLE 55
Model 3 Blinks Fixed Effects Test.
Type III Tests of Fixed Effects^{a}

| Source | Numerator df | Denominator df | F | Sig. |
|---|---|---|---|---|
| Intercept | 1 | .000 | 15.732 | .000 |
| Med_type | 3 | 80 | 1.418 | .244 |

^{a}Dependent Variable: Blinks.

Blinks were not found to be statistically significant between the ADHD medication types.

TABLE 56
Model 3 Blink Rate Fixed Effects Test.
Type III Tests of Fixed Effects^{a}

| Source | Numerator df | Denominator df | F | Sig. |
|---|---|---|---|---|
| Intercept | 1 | .000 | 4.592 | 1.000 |
| Med_type | 3 | 80 | 1.296 | .282 |

^{a}Dependent Variable: BlinkRate.

Blink rate was not found to be statistically significant between the ADHD medication types.

TABLE 57
Model 3 Area Under Curve (AUC) Fixed Effects Test.
Type III Tests of Fixed Effects^{a}

| Source | Numerator df | Denominator df | F | Sig. |
|---|---|---|---|---|
| Intercept | 1 | 80 | 53.795 | <.001 |
| Med_type | 3 | 80 | 2.441 | 070 |

^{a}Dependent Variable: AreaUnderCurve.

AUC was not found to be statisticaly significant between the ADHD medication types.

TABLE 58
Model 3 Max Closing Velocity Fixed Effects Test.
Type III Tests of Fixed Effects^{a}

| Source | Numerator df | Denominator df | F | Sig. |
|---|---|---|---|---|
| Intercept | 1 | 1214.747 | 134.799 | <.001 |
| Med_type | 3 | 80.000 | 5.159 | 003 |

^{a}Dependent Variable: MaxClosingVelocity.

TABLE 59
Model 3 Max Closing Velocity Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 6.624 | .686 | 1694.848 | 9.658 | <.001 | 5.278 | 7.969 |
| [Med_type = 1] | 2.151 | 1.011 | 80 | 2.128 | .036 | .140 | 4.163 |
| [Med_type = 2] | 2.659 | 1.011 | 80 | 2.630 | .010 | .647 | 4.671 |
| [Med_type = 3] | 1.378 | .441 | 80 | 3.128 | .002 | .501 | 2.255 |
| [Med_type = 4] | 0^{b} | 0 | . | . | . | . | . |

^{a}Dependent Variable: MaxClosingVelocity.
^{b}This parameter is set to zero because it is redundant.

Max closing velocity was found to be statistically significant between all ADHD medication types that were tested.

TABLE 60
Model 3 Max Opening Velocity Fixed Effects Test.
Type III Tests of Fixed Effects^{a}

| Source | Numerator df | Denominator df | F | Sig. |
|---|---|---|---|---|
| Intercept | 1 | .000 | 9.293 | 1.000 |
| Med_type | 3 | 80.000 | 1.433 | .239 |

^{a}Dependent Variable: MaxOpeningVelocity.

Max Opening Velocity was not found to be statistically significant between the ADHD medication types.

TABLE 61
Model 3 Excursions Fixed Effects Test.
Type III Tests of Fixed Effects^{a}

| Source | Numerator df | Denominator df | F | Sig. |
|---|---|---|---|---|
| Intercept | 1 | 19151.518 | 59.847 | <.001 |
| Med_type | 3 | 80.000 | 3.783 | 0.014 |

^{a}Dependent Variable: Excursions.

TABLE 62
Model 3 Excursions Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | 102.184 | 14.608 | 28880.000 | 6.995 | <.001 | 73.553 | 130.816 |
| [Med_type = 1] | 17.606 | 10.598 | 80.000 | 1.661 | 0.101 | −3.484 | 38.695 |
| [Med_type = 2] | 13.866 | 10.598 | 80.000 | 1.308 | 0.194 | −7.224 | 34.955 |
| [Med_type = 3] | 14.707 | 4.619 | 80.000 | 3.184 | 0.002 | 5.514 | 23.900 |
| [Med_type = 4] | 0^{b} | 0 | . | . | . | . | . |

^{a}Dependent Variable: Excursions.
^{b}This parameter is set to zero because it is redundant.

Excursions were found to be statistically significant between ADHD medication types, particularly MPH. In conclusion, Model 3 demonstrated that Delta 30, Max Closing Velocity, and Excursion parameters were identified to be statistically predictive between ADHD treatment types. Blink reflex parameters were particularly predictive for patients taking MPH, but the change was likely driven from the small sample size (4 participants (ATX=2, MPH=2), and was not predictive for the larger group of 20 AMP patients.

Differential latency was close to being predictive, but the change was driven from 4 participants (ATX=2, MPH =2) and not AMP. Delta 30, which showed predictiveness in the other models, was also predictive in this model.

Predictive significance for blink reflex parameters in determining ADHD treatment efficacy is largely driven by AMP and MPH, which are both stimulant medications. The effect of these medications caused a slight increase in initial velocity, which is explained clinically from the drug's mechanism of action. The effect of medication was also determined to be significant on excursion (eyelid travel distance).

The most significant blink parameter for predicting ADHD medication adherence is Max Closing Velocity. All three medications were significantly associated with an increase in max closing velocity compared to controls and participants before taking their ADHD Medications.

Further analysis was performed to determine if the variables are affected by sleep, urine specific gravity (dehydration), and age were possible confounding variables (summarized in TABLES 63-66). We performed a statistical model that performed while taking into account medication type, the aforementioned three confounders, a random effect, and the repeated measure of time.

TABLE 63
Further Analysis of Max Closing Velocity.
TYpe III Tests of Fixed Effects^{a}

| Source | Numerator df | Denominator df | F | Sig. |
|---|---|---|---|---|
| Intercept | 1 | 0.000 | 0.165 | 1.000 |
| Med_type | 3 | 71.000 | 4.221 | 0.008 |
| HoursofSleep | 1 | 71.000 | 0.168 | 0.683 |
| UrineSpecificGravity | 1 | 71.000 | 0.845 | 0.361 |
| Age_years | 1 | 71.000 | 3.422 | 0.068 |

^{a}Dependent Variable: MaxClosingVelocity

TABLE 64
Model 3 Excursions Estimates of Fixed Effects.
Estimates of Fixed Effects^{a}

| | | Std. | | | | 95% Confidence Interval | |
|---|---|---|---|---|---|---|---|
| Parameter | Estimate | Error | df | t | Sig. | Lower Bound | Upper Bound |
| Intercept | −14.308 | 31.505 | .000 | −.454 | 1.000 | −764590.433 | 764561.816 |
| [Med_type = 1] | 2.477 | 1.057 | 71.000 | 2.343 | .022 | .369 | 4.584 |
| [Med_type = 2] | 2.277 | 1.048 | 71.000 | 2.174 | .033 | .189 | 4.366 |
| [Med_type = 3] | 1.265 | .473 | 71.000 | 2.674 | .009 | .322 | 2.208 |
| [Med_type = 4] | 0^{b} | 0 | . | . | . | . | . |
| HoursofSleep | −.074 | .181 | 71.000 | −.409 | .683 | −.434 | .286 |
| UrineSpecificGravity | 28.621 | 31.135 | 71.000 | .919 | .361 | −33.462 | 90.703 |
| Age_years | −.362 | .196 | 71.000 | −1.850 | .068 | −.752 | .028 |

^{a}Dependent Variable: MaxClosingVelocity
^{b}This parameter is set to zero because it is redundant.

Even after controlling for possible confounding variables, the main effect of medication type, as well as each individual ADHD medication, demonstrated a statistically significant effect on Max Closing Velocity.

### Paired T-Test Between ADHD Group Before and After Medications

Statistical analyses within each patient-paired pre-medication to post-medication set of observations in the ADHD group revealed that certain blink reflex parameters hold higher significance with determining effects of the medication (e.g., as summarized in TABLE 65).

TABLE 65
Patient-Paired Sample test for Blink Reflex Parameters
Paired Samples Test

| | Paired Differences | | | | | | | |
|---|---|---|---|---|---|---|---|---|
| | | Std. | 95% Confidence Interval | | | | Significance | |
| | | Error | of the Difference | | | | One- | Two- |
| | Mean ± S.D. | Mean | Lower | Upper | t | df | Sided p | Sided p |
| Latency | 0.78167 ± 7.06516 | 1.44217 | −2.20169 | 3.76502 | 0.542 | 23 | 0.297 | 0.593 |
| Differential Latency | 0.36583 ± 2.61181 | 0.53313 | −0.73704 | 1.46870 | 0.686 | 23 | 0.250 | 0.499 |
| Delta30 | 0.35083 ± 2.12779 | 0.43433 | −0.54765 | 1.24932 | 0.808 | 23 | 0.214 | 0.428 |
| Initial Velocity | −0.03250 ± 0.51115 | 0.10434 | −0.24834 | 0.18334 | −0.311 | 23 | 0.379 | 0.758 |
| Time to Close | 0.78833 ± 2.87098 | 0.58604 | −0.42397 | 2.00064 | 1.345 | 23 | 0.096 | 0.192 |
| Time to Open | 53.26375 ± 216.61251 | 44.21584 | −38.20369 | 144.73119 | 1.205 | 23 | 0.120 | 0.241 |
| Time under 20 | 10.30875 ± 63.23735 | 12.90827 | −16.39404 | 37.01154 | 0.799 | 23 | 0.216 | 0.433 |
| Oscillations | 0.77167 ± 2.86399 | 0.58461 | −0.43769 | 1.98102 | 1.320 | 23 | 0.100 | 0.200 |
| Blinks | 0.42125 ± 1.63416 | 0.33357 | −0.26880 | 1.11130 | 1.263 | 23 | 0.110 | 0.219 |
| Blink Rate | 8.31708 ± 25.78790 | 5.26393 | −2.57219 | 19.20636 | 1.580 | 23 | 0.064 | 0.128 |
| Area Under Curve | 4704.810 ± 22068.630 | 4504.74042 | −4613.9547 | 14023.5763 | 1.044 | 23 | 0.154 | 0.307 |
| Max Closing Velocity | −0.17458 ± 1.80312 | 0.36806 | −0.93597 | 0.58681 | −0.474 | 23 | 0.320 | 0.640 |
| Max Opening Velocity | 0.21417 ± 0.90892 | 0.18553 | −0.16964 | 0.59797 | 1.154 | 23 | 0.130 | 0.260 |
| Excursions | −0.19917 ± 16.68504 | 3.40582 | −7.24464 | 6.84631 | −0.058 | 23 | 0.477 | 0.954 |

ADHD patients demonstrated significant differences from patients without ADHD, even irrespective of medications. The ADHD cohort took longer to open their eyes (i.e., time to open) after the blink stimulus, despite the fact that they exhibited faster initial velocity. Additionally, unmedicated patients exhibited a greater area under the curve. Therefore, it is statistically useful to use the blink reflex to diagnose ADHD prior to medication use.

Pharmacological intervention in the ADHD cohort demonstrated an effect on the blink reflex parameters. The largest effect of ADHD medications was on Max Closing Velocity, where all three ADHD medication types (amphetamines, methylphenidates, and atomoxetine) were associated with an increase in max closing velocity, even when controlling for sleep duration the night prior and hydration levels.

Overall, with respect to diagnosing ADHD, while all parameters hold some clinical value, the most significant differences were observed in the following variables:
1. Delta 30 (p=0.019);
2. Initial Velocity (p=0.004);
3. Time to Open (p=0.003);
4. Area Under Curve (p<0.001);
5. Max Closing Velocity (p =0.008); and
6. Excursion (p =0.006).

For future studies, TABLE 66 details the estimated sample sizes required to more accurately predict ADHD diagnosis and treatment response, with Delta 30 being the largest total sample size and Max Closing Velocity the highest.

TABLE 66
Sample Size Estimation.

| | | | | | Total |
|---|---|---|---|---|---|
| | Power | Significance | N per | N per group after | sample |
| Variable | (β) | Level (α) | group | 15% attrition | size |
| Delta 30 | 0.80 | 0.05 | 36 | 42 | 84 |
| Delta 30 | 0.90 | 0.05 | 48 | 53 | 106 |
| Delta 30 | 0.95 | 0.05 | 59 | 68 | 136 |
| Max Closing | 0.80 | 0.05 | 32 | 37 | 74 |
| Velocity | | | | | |
| Max Closing | 0.90 | 0.05 | 43 | 50 | 100 |
| Velocity | | | | | |
| Max Closing | 0.95 | 0.05 | 52 | 60 | 120 |
| Velocity | | | | | |

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. (canceled)

2. A method of diagnosing and treating a patient afflicted with attention-deficit/hyperactivity disorder (ADHD), comprising:
(a) measuring a blink reflex in a patient suspected to be afflicted with ADHD, the measurement comprising:
(i) applying a non-electrical stimulus to an eye of the patient to induce a blink reflex and
(ii) assessing one or more blink-associated parameters;
(b) diagnosing the patient as afflicted with ADHD or not afflicted with ADHD by comparing the one or more blink-associated parameters to equivalent one or more blink-associated parameters from a patient who is not afflicted with ADHD; and
(c) administering an effective amount of an ADHD treatment, optionally a medication, to a patient diagnosed as afflicted with ADHD.

3. A method of selecting an attention-deficit/hyperactivity disorder (ADHD) treatment for a patient afflicted with ADHD, comprising:
(a) measuring a blink reflex in the patient afflicted with ADHD, the measurement comprising:
(i) applying a non-electrical stimulus to an eye of the patient to induce a blink reflex and
(ii) assessing one or more blink-associated parameters; and
(b) selecting an ADHD treatment for the patient afflicted with ADHD by comparing the one or more blink-associated parameters to equivalent one or more blink-associated parameters from the patient when receiving the ADHD treatment.

4. (canceled)

5. The method of claim 2, wherein the diagnosing comprises:
diagnosis of disease onset;
determination of disease stage and/or severity;
determination of disease progress; and/or
determination of cognitive state and/or mental capacity.

6-9. (canceled)

10. The method of claim 2, wherein the method measures latency until physiological response to stimulus.

11. The method of claim 2, wherein the blink reflex correlates with functionality of one or more of the trigeminal and facial nerves.

12. The method of claim 2, wherein the non-electrical stimulus is one or more physical stimuli.

13. The method of claim 2, wherein the one or more physical stimuli comprises a puff of air, a flash of light, a noise, and/or a vibration.

14-15. (canceled)

16. The method of claim 2, wherein the non-electrical stimulus is compressed air and is applied across the eye, and/or wherein the non-electrical stimulus is applied to the canthus or outer canthus.

17. (canceled)

18. The method of claim 2, wherein the blink reflex is measured with a high-speed camera, is measured by capturing and analyzing high-definition video, and/or is measured with a blink reflex monitoring device, wherein the blink reflex monitoring device is a wearable device or a handheld device.

19. (canceled)

20. The method of claim 2, wherein the blink reflex is not measured with an electromyography (EMG) sensor or device.

21-23. (canceled)

24. The method of claim 2, wherein the blink reflex monitoring device has or comprises:
a front end, a back end, a first plane extending through the front end and the back end,
a second plane intersecting the first plane between the front end and the back end, the back end configured to receive a portion of a face of a user and disposed opposite the front end; and
a strap coupled to the blink reflex monitoring device and having a first position and a second position,
wherein in the first position the strap extends away from the blink reflex monitoring device along the first plane end and is configured to secure the blink reflex monitoring device to a head of a user and in the second position the strap extends below the blink reflex monitoring device along the second plane and is configured to support the blink reflex monitoring device above a surface.

25. The method of claim 2, wherein the blink reflex monitoring device has or comprises:
a front end and a back end, the back end configured to receive a portion of a face of a user and disposed opposite the front end;
at least one strap coupled to the blink reflex monitoring device proximate the back end, the at least one strap configured to be secured around a head of a user; and
an air compression system including a source of compressed air and a valve, the air compression system disposed within the blink reflex monitoring device and the valve configured to output compressed air from the back end at a pressure of about 3 PSI to about 60 PSI.

26. The method of claim 25, wherein the air compression system disposed within the blink reflex monitoring device and the valve configured to output compressed air from the back end at a pressure of about 30 PSI to about 50 PSI.

27-29. (canceled)

30. The method of claim 2, wherein the measuring a blink reflex, comprises:
providing a blink reflex monitoring device to a user, the blink reflex monitoring device having an air compression system including a compressed air source and a valve;
outputting a first burst of compressed air from the compressed air source through the valve at a first pressure;
detecting a presence or an absence of a blink in response to the first burst of compressed air source at the first pressure;
if the blink is present, then measuring a duration between the output of the first burst of compressed air at the first pressure and the blink to determine the blink reflex; and
if the blink is absent, then outputting a second burst of compressed air from the compressed air source at a second pressure, the second pressure being greater than the first pressure and measuring a duration between the output of the second burst of compressed air at the second pressure and the blink to determine the blink reflex.

31. The method of claim 2, wherein the one or more blink-associated parameters are selected from:
(a) latency, optionally in milliseconds, comprising a time differential between stimulation and eyelid movement, e.g., one or more of upper eyelid or lower eyelid;
(b) differential latency, optionally in milliseconds, comprising a time differential between the start of ipsilateral eye movement and the start of contralateral eye movement;
(c) delta 30, comprising a time difference between ipsilateral eye and contralateral eye movement;
(d) eyelid excursion, optionally in pixels, comprising a distance traveled by the eyelid from the tonic lid position to closed position;
(e) initial lid velocity, optionally in pixels/msec, comprising an average eyelid speed following start of eyelid movement, e.g. the first about 5 frames, the first about 7 frames, or the first about 10 frames;
(f) time to close, optionally in log scale, comprising a time for lid to travel from tonic lid position to the closed position;
(g) time to open, optionally in log scale, comprising a time for lid to travel from closed position back to tonic lid position;
(h) time under threshold, optionally in log scale, comprising a time that the eyelid spends below the threshold position;
(i) number of oscillations, comprising cycles of up and down upper eyelid movement after a stimulated blink;
(j) total blink time, optionally in log scale, comprising a time from start of eyelid movement until it returns to its tonic lid position;
(k) number of blinks;
(l) blink rate, optionally per minute;
(m) area under curve, optionally in pixels;
(n) maximum closing velocity, optionally in milliseconds, comprising a maximum velocity during eyelid closure;
(o) maximum opening velocity, optionally in milliseconds, comprising a maximum velocity during eyelid opening; and
(p) time to first oscillation, optionally in log scale, comprising the elapsed time between the eyelid returning within threshold of a tonic position for a stimulated blink and the onset of the next unstimulated blink.

32. (canceled)

33. The method of claim 31, wherein the one or more blink-associated parameters are used for diagnosing ADHD and/or determining adherence to an ADHD treatment.

34-39. (canceled)

40. The method of claim 2, wherein the patient afflicted with ADHD, in comparison to a patient that is not afflicted with ADHD, exhibits one or more of:
decreased latency;
decreased differential latency;
decreased delta 30;
increased eyelid excursion;
increased initial lid velocity;
increased time to close;
increased time to open;
increased time under threshold;
increased number of oscillations;
increased increaed total blink time;
increased number of blinks;
increased blink rate;
increased increaed area under curve;
increased maximum closing velocity;
increased increaed maximum opening velocity; and
increased time to first oscillation.

41-46. (canceled)

47. The method of claim 2, wherein the ADHD is diagnosed by Diagnostic and Statistical Manual of Mental Disorders (DSM-5) criteria, and/or wherein the ADHD comprises one or more symptoms selected from being easily distracted, forgetful, daydreaming, disorganization, poor concentration, difficulty completing tasks, excessive fidgetiness and restlessness, hyperactivity, difficulty waiting and remaining seated, immature behavior, and/or destructive behavior.

48. (canceled)

49. The method of claim 2, wherein the patient is experiencing symptoms associated with combined type ADHD, symptoms associated with predominantly inattentive type ADHD, and/or symptoms associated with predominantly hyperactive-impulsive type ADHD.

50-53. (canceled)

54. The method of claim 2, wherein the method further comprises prescribing an ADHD treatment as a function of the blink reflex, and/or wherein prescribing comprises changing a prescription from a first ADHD medication to a second ADHD medication, and/or wherein the method further comprises determining a level of adherence to ADHD treatment as a function of the blink reflex.

55-56. (canceled)
